(19) United States
(12) Patent Application Publication
(10) Pub. No.: US 2015/0372349 A1
(43) Pub. Date: Dec. 24, 2015

(54) NON-AQUEOUS ELECTROLYTE AND ELECTRICITY STORAGE DEVICE

(71) Applicant: UBE INDUSTRIES, LTD., Ube-shi (JP)

(72) Inventor: Shoji SHIKITA, Ube-shi (JP)

(73) Assignee: UBE INDUSTRIES, LTD., Ube-shi (JP)

(21) Appl. No.: 14/760,825

(22) PCT Filed: Jan. 20, 2014
(86) PCT No.: PCT/JP2014/051020

§ 371 Date: Jul. 14, 2015

(30) Foreign Application Priority Data
Jan. 23, 2013 (JP) 2013-009713

(51) Int. Cl. H01M 10/0567; H01M 10/052; C07C 69/96; C07C 309/73; C07C 69/22; C07C 69/40; C07F 9/09; C07C 309/66; H01M 10/0569; C07C 69/12

(52) U.S. Cl. CPC H01M 10/0567; H01M 10/0569; H01M 10/052; C07C 69/96; C07C 69/12; C07C 69/22; C07C 69/40; C07F 9/09; C07C 309/66; C07C 309/73; H01M 2300/0028; H01M 2300/0037; H01M 2300/0025; H01M 2220/30

(57) ABSTRACT

The present invention provides a nonaqueous electrolytic solution capable of improving electrochemical characteristics in the case of using an energy storage device at a high temperature and further capable of inhibiting the gas generation as well as a capacity retention rate after a high-temperature cycle, and also to provide an energy storage device using the same. Disclosed are a nonaqueous electrolytic solution having an electrolyte salt dissolved in a nonaqueous solvent, the nonaqueous electrolytic solution containing a phenyl ester compound represented by the following general formula (I), and an energy storage device.

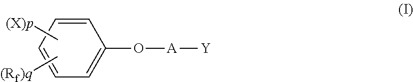

(In the formula, R_{f} represents a fluoroalkyl group having 1 to 6 carbon atoms; X represents a halogen atom; each of p and q is an integer of 1 to 4; and (p+q) is 5 or less. A has a structure represented by —S(═O)₂—, —C(═O)—, —C(═O)—O—, —C(═O)-L¹-C(═O)—, —C(═O)-L²-P(═O)(OR)—O—, or —P(═O)(OR)—O—. Y represents a fluorine atom, a hydrogen atom, an alkyl group, an alkenyl group, an alkynyl group, or an aryl group; L¹ represents an alkylene group, an alkenylene group, an alkynylene group, or a direct bond; L² represents an alkylene group; and R represents an alkyl group. However, only when A is —S(═O)₂—, Y may be a fluorine atom; and only when A is —C(═O)—, Y may be a hydrogen atom. At least one hydrogen atom in each group of the aforementioned alkyl group, alkenyl group, alkynyl group, aryl group, alkylene group, alkenylene group, and alkynylene group may be substituted with a halogen atom.)

### TECHNICAL FIELD

The present invention relates to a nonaqueous electrolytic solution capable of improving electrochemical characteristics on the occasion of using an energy storage device at a high temperature and also an energy storage device using the same.

### BACKGROUND ART

An energy storage device, especially a lithium secondary battery, has been widely used recently for a power source of an electronic device, such as a mobile telephone, a notebook personal computer, etc., and a power source for an electric vehicle or electric power storage. With respect to a thin electronic device, such as a tablet device, an ultrabook, etc., a laminate-type battery or a prismatic battery using an aluminum laminate film, an aluminum alloy, or the like for an outer packaging member thereof is frequently used. In such a battery, the outer packaging member is thin, and therefore, there is involved such a problem that the battery is easily deformed, so that the deformation very likely influences the electronic device.

A lithium secondary battery is mainly constituted of a positive electrode and a negative electrode, each containing a material capable of absorbing and releasing lithium, and a nonaqueous electrolytic solution containing a lithium salt and a nonaqueous solvent; and a carbonate, such as ethylene carbonate (EC), propylene carbonate (PC), etc., is used as the nonaqueous solvent.

In addition, a lithium metal, a metal compound capable of absorbing and releasing lithium (e.g., a metal elemental substance, a metal oxide, an alloy with lithium, etc.), and a carbon material are known as the negative electrode of the lithium secondary battery. In particular, a nonaqueous electrolytic solution secondary battery using, as the carbon material, a carbon material capable of absorbing and releasing lithium, for example, coke or graphite (e.g., artificial graphite or natural graphite), etc., is widely put into practical use. Since the aforementioned negative electrode material stores/releases lithium and an electron at an extremely electronegative potential equal to the lithium metal, it has a possibility that a lot of solvents are subjected to reductive decomposition, and a part of the solvent in the electrolytic solution is reductively decomposed on the negative electrode regardless of the kind of the negative electrode material, so that there were involved such problems that the movement of a lithium ion is disturbed due to deposition of decomposition products, generation of a gas, or expansion of the electrode, thereby worsening battery characteristics, such as cycle property, etc., especially in the case of using the battery at a high temperature; and that the battery is deformed due to expansion of the electrode. Furthermore, it is known that a lithium secondary battery using a lithium metal or an alloy thereof, a metal elemental substance, such as tin, silicon, etc., or a metal oxide thereof as the negative electrode material may have a high initial battery capacity, but the battery capacity and the battery performance thereof, such as the cycle property, may be largely worsened because the micronized powdering of the material may be promoted during cycles, which brings about accelerated reductive decomposition of the nonaqueous solvent, as compared with the negative electrode formed of a carbon material, and the battery may be deformed due to expansion of the electrode.

Meanwhile, since a material capable of absorbing and releasing lithium, which is used as a positive electrode material, such as LiCoO₂, LiMn₂O₄, LiNiO₂, LiFePO₄, etc., stores and releases lithium and an electron at an electropositive voltage of 3.5 V or more on the lithium basis, it has a possibility that a lot of solvents are subjected to oxidative decomposition especially in the case of using the battery at a high temperature, and a part of the solvent in the electrolytic solution is oxidatively decomposed on the positive electrode regardless of the kind of the positive electrode material, so that there were involved such problems that the resistance is increased due to deposition of decomposition products; and that a gas is generated due to decomposition of the solvent, thereby expanding the battery.

Irrespective of the foregoing situation, the multifunctionality of electronic devices on which lithium secondary batteries are mounted is more and more advanced, and power consumption tends to increase. The capacity of the lithium secondary battery is thus being much increased, and the electrolytic solution is in the environment where decomposition is apt to more likely occur because of a temperature increase of the battery due to heat generation from the electronic device, a high voltage of set charge voltage of the battery, and the like. In addition, because of an increase of a density of the battery, a reduction of a useless space capacity within the battery, and so on, a volume occupied by the nonaqueous electrolytic solution in the battery is becoming small. In consequence, it is the present situation that in the case of using the battery at a high temperature, the battery performance is apt to be lowered by decomposition of a bit nonaqueous electrolytic solution, and a problem that the battery becomes unable to be used due to expansion of the battery caused by the gas generation, actuation of a safety mechanism to cut off the current, etc., or the like occurs.

PTL 1 describes that when an electrolytic solution including a phenyl ester compound, such as 4-(trifluoromethyl)phenyl acetate and 3,4-difluorophenyl acetate, is used, not only overcharge properties of a lithium secondary battery can be improved, but also storage properties and continuous charge properties can be improved.

PTL 2 describes that when an electrolytic solution including a phenyl sulfonate compound, such as 2,4-difluorophenyl methanesulfonate, is used, a low-temperature cycle property of a battery can be improved.

PTL 3 describes that when an electrolytic solution including a phenyl sulfonate compound, such as 2-trifluoromethylphenyl methanesulfonate, is used, a lithium battery having excellent electrochemical characteristics over a wide temperature range is obtained.

PTL 1: WO 2011/025016

PTL 2: WO 2009/057515

PTL 3: WO 2012/144306

### SUMMARY OF INVENTION

### Technical Problem

Problems to be solved by the present invention are to provide a nonaqueous electrolytic solution capable of improving electrochemical characteristics in the case of using an energy storage device at a high temperature and further capable of inhibiting the gas generation as well as a discharge capacity retention rate after a high-voltage cycle, and also to provide an energy storage device using the same.

### Solution to Problem

The present inventor made extensive and intensive investigations regarding the performance of the nonaqueous electrolytic solutions of the aforementioned conventional technologies. As a result, according to the nonaqueous electrolytic solutions of the above-cited PTLs 1 to 3, in the case of contemplating to widen a use temperature of the energy storage device, it may not be said that the nonaqueous electrolytic solutions of PTLs 1 to 3 are thoroughly satisfactory. Above all, PTLs 1 to 3 do not disclose anything for the problems of improving the charge/discharge cycle property in the case of using the energy storage device at a high temperature and inhibiting the gas generation following charge/discharge at all.

Then, in order to solve the above-described problem, the present inventor made extensive and intensive investigations. As a result, it has been found that by adding a phenyl ester compound in which a specified benzene ring is substituted with both a halogen atom and a fluoroalkyl group to a nonaqueous electrolytic solution, not only a capacity retention rate after a cycle in the case of using an energy storage device at a high temperature can be improved, but also the gas generation can be inhibited, leading to accomplishment of the present invention.

Specifically, the present invention provides the following (1) to (3).

(1) A nonaqueous electrolytic solution having an electrolyte salt dissolved in a nonaqueous solvent, the nonaqueous electrolytic solution containing a phenyl ester compound represented by the following general formula (I), in which the benzene ring is substituted with both a halogen atom and a fluoroalkyl group.

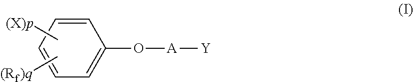

(In the formula, R_{f} represents a fluoroalkyl group having 1 to 6 carbon atoms; X represents a halogen atom; each of p and q is an integer of 1 to 4; and (p+q) is 5 or less. A has a structure represented by —S(═O)₂—, —C(═O)—, —C(═O)—O—, —C(═O)-L¹-C(═O)—, —C(═O)-L²-P(═O)(OR)—O—, or —P(═O)(OR)—O—. Y represents a fluorine atom, a hydrogen atom, an alkyl group having 1 to 6 carbon atoms, an alkenyl group having 2 to 6 carbon atoms, an alkynyl group having 3 to 6 carbon atoms, or an aryl group having 6 to 12 carbon atoms; L¹ represents an alkylene group having 1 to 8 carbon atoms, an alkenylene group having 2 to 8 carbon atoms, an alkynylene group having 2 to 8 carbon atoms, or a direct bond; L² represents an alkylene group having 1 to 8 carbon atoms; and R represents an alkyl group having 1 to 6 carbon atoms. However, only when A is —S(═O)₂—, Y may be a fluorine atom; and only when A is —C(═O)—, Y may be a hydrogen atom.

At least one hydrogen atom in each group of the aforementioned alkyl group, alkenyl group, alkynyl group, aryl group, alkylene group, alkenylene group, and alkynylene group may be substituted with a halogen atom.)

(2) An energy storage device including a positive electrode, a negative electrode, and a nonaqueous electrolytic solution having an electrolyte salt dissolved in a nonaqueous solvent, the nonaqueous electrolytic solution containing the phenyl ester compound represented by the foregoing formula (I).
(3) A phenyl ester compound represented by the following general formula (II), in which the benzene ring is substituted with both a halogen atom and a fluoroalkyl group.

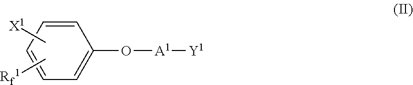

(In the formula, R_{f}¹ represents a fluoroalkyl group having 1 to 6 carbon atoms; and X¹ represents a halogen atom. A¹ has a structure represented by —S(═O)₂—, —C(═O)—, —C(═O)—O—, —C(═O)-L³-C(═O)—, —C(═O)-L⁴-P(═O)(OR¹)—O—, or —P(═O)(OR¹)—O—. Y¹ represents a fluorine atom, a hydrogen atom, an alkyl group having 1 to 6 carbon atoms, an alkenyl group having 2 to 6 carbon atoms, an alkynyl group having 3 to 6 carbon atoms, or an aryl group having 6 to 12 carbon atoms; L³ represents an alkylene group having 1 to 8 carbon atoms, an alkenylene group having 2 to 8 carbon atoms, an alkynylene group having 2 to 8 carbon atoms, or a direct bond; L⁴ represents an alkylene group having 1 to 8 carbon atoms; and R¹ represents an alkyl group having 1 to 6 carbon atoms. However, only when A¹ is —S(═O)₂—, Y may be a fluorine atom; only when A¹ is —C(═O)—, Y may be a hydrogen atom, and the case where A¹ is —S(═O)₂— and Y¹ is a trifluoromethyl group is excluded.

At least one hydrogen atom in each group of the aforementioned alkyl group, alkenyl group, alkynyl group, aryl group, alkylene group, alkenylene group, and alkynylene group may be substituted with a halogen atom.)

### Advantageous Effects of Invention

According to the present invention, it is possible to provide a nonaqueous electrolytic solution capable of not only improving a capacity retention rate after a cycle but also inhibiting the gas generation in the case of using an energy storage device at a high temperature, and also to provide an energy storage device, such as a lithium battery, etc., using the same.

### DESCRIPTION OF EMBODIMENTS

### [Nonaqueous Electrolytic Solution]

The nonaqueous electrolytic solution of the present invention is a nonaqueous electrolytic solution having an electrolyte dissolved in a nonaqueous solvent, the nonaqueous solvent containing a phenyl ester compound represented by the foregoing general formula (I), in which the benzene ring is substituted with both a halogen atom and a fluoroalkyl group.

Although the reason why the nonaqueous electrolytic solution of the present invention is capable of significantly improving the electrochemical characteristics in the case of using an energy storage device at a high temperature has not always been elucidated yet, the following may be considered.

The phenyl ester compound represented by the foregoing general formula (I) has a functional group with high electrophilicity, such as an alkanesulfonyl group, an alkylcarbonyl group, an alkoxycarbonyl group, etc., and a phenyl group having not only a fluoroalkyl group that is an electron-withdrawing group which is bulky and does not leave but also a halogen atom that is a strong electron-withdrawing group. In view of the fact that the phenyl ester compound has the functional group with high electrophilicity and the electron-withdrawing groups, decomposability of the compound is improved, and the benzene rings are polymerized with each other on a negative electrode, thereby forming a benzene ring-originated surface film with high heat resistance. Furthermore, since the fluoroalkyl group is a substituent which is bulky and does not leave, excessive polymerization is inhibited. In consequence, it may be considered that a remarkable improvement of the high-temperature cycle property, which is never attained by a compound having only a bulky and electron-withdrawing substituent, for example, 4-(trifluoromethyl)phenyl acetate, or a compound having only a strong electron-withdrawing group, for example, 2,4-difluorophenyl acetate, has been obtained.

The compound which is contained in the nonaqueous electrolytic solution of the present invention is represented by following general formula (I).

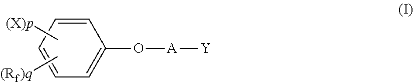

(In the formula, R_{f} represents a fluoroalkyl group having 1 to 6 carbon atoms; X represents a halogen atom; each of p and q is an integer of 1 to 4; and (p+q) is 5 or less. A has a structure represented by —S(═O)₂—, —C(═O)—, —C(═O)—O—, —C(═O)-L¹-C(═O)—, —C(═O)-L²-P(═O)(OR)—O—, or —P(═O)(OR)—O—. Y represents a fluorine atom, a hydrogen atom, an alkyl group having 1 to 6 carbon atoms, an alkenyl group having 2 to 6 carbon atoms, an alkynyl group having 3 to 6 carbon atoms, or an aryl group having 6 to 12 carbon atoms; L¹ represents an alkylene group having 1 to 8 carbon atoms, an alkenylene group having 2 to 8 carbon atoms, an alkynylene group having 2 to 8 carbon atoms, or a direct bond; L² represents an alkylene group having 1 to 8 carbon atoms; and R represents an alkyl group having 1 to 6 carbon atoms. However, only when A is —S(═O)₂—, Y may be a fluorine atom; and only when A is —C(═O)—, Y may be a hydrogen atom.

At least one hydrogen atom in each group of the aforementioned alkyl group, alkenyl group, alkynyl group, aryl group, alkylene group, alkenylene group, and alkynylene group may be substituted with a halogen atom.)

In the foregoing general formula (I), X represents a halogen atom, and as specific examples of X, a fluorine atom, a chlorine atom, or a bromine atom is suitably exemplified. Of these, a fluorine atom or a chlorine atom is more preferred, and a fluorine atom is still more preferred.

In the foregoing general formula (I), R_{f} represents a fluoroalkyl group having 1 to 6 carbon atoms, in which at least one hydrogen atom is substituted with a fluorine atom, and R_{f} is more preferably a fluoroalkyl group having 1 or 2 carbon atoms, and still more preferably a fluoroalkyl group having one carbon atom.

As specific examples of the fluoroalkyl group as R_{f}, there are suitably exemplified a fluoromethyl group, a difluoromethyl group, a trifluoromethyl group, a 2,2,2-trifluoroethyl group, a perfluoroethyl group, a perfluoropropyl group, a perfluorobutyl group, and the like. Of these, fluoroalkyl groups having 1 or 2 carbon atoms, such as a difluoromethyl group, a trifluoromethyl group, a 2,2,2-trifluoroethyl group, a perfluoroethyl group, etc., are preferred, and fluoroalkyl groups having one carbon atom, such as a difluoromethyl group, a trifluoromethyl group, etc., are more preferred.

In the foregoing general formula (I), each of p and q represents an integer of 1 to 4, and (p+q) is 5 or less. Each of p and q is more preferably 1 to 2, and still more preferably 1.

In the foregoing general formula (I), A is preferably —S(═O)₂—, —C(═O)—O—, —C(═O)-L¹-C(═O)—, or —C(═O)-L²-P(═O)(OR)—O—, and more preferably —S(═O)₂— or —C(═O)—O—.

In the foregoing general formula (I), Y is preferably a fluorine atom, a hydrogen atom, an alkyl group having 1 to 4 carbon atoms, in which at least one hydrogen atom may be substituted with a halogen atom, an alkenyl group having 2 to 5 carbon atoms, in which at least one hydrogen atom may be substituted with a halogen atom, an alkynyl group having 3 to 6 carbon atoms, in which at least one hydrogen atom may be substituted with a halogen atom, or an aryl group having 6 to 10 carbon atoms, in which at least one hydrogen atom may be substituted with a halogen atom, and more preferably a fluorine atom, a hydrogen atom, an alkyl group having 1 to 3 carbon atoms, an alkenyl group having 2 to 4 carbon atoms, an alkynyl group having 3 to 5 carbon atoms, or an aryl group having 6 to 8 carbon atoms, in which at least one hydrogen atom may be substituted with a halogen atom.

In particular, in the case where A is —C(═O)-L²-P(═O)(OR)—O— or —P(═O)(OR)—O—, an alkyl group having 1 to 3 carbon atoms is preferable.

L¹ is preferably an alkylene group having 2 to 7 carbon atoms, in which at least one hydrogen atom may be substituted with a halogen atom, an alkenylene group having 2 to 6 carbon atoms, in which at least one hydrogen atom may be substituted with a halogen atom, an alkynylene group having 2 to 6 carbon atoms, in which at least one hydrogen atom may be substituted with a halogen atom, or a direct bond (no substituent), and more preferably an alkylene group having 2 to 7 carbon atoms, an alkenylene group having 2 to 6 carbon atoms, an alkynylene group having 2 to 6 carbon atoms, or a direct bond.

L² is preferably an alkylene group having 1 to 4 carbon atoms, in which at least one hydrogen atom may be substituted with a halogen atom, and more preferably an alkylene group having 1 or 2 carbon atoms, in which at least one hydrogen atom may be substituted with a halogen atom.

R is preferably an alkyl group having 1 to 4 carbon atoms, in which at least one hydrogen atom may be substituted with a halogen atom, and more preferably an alkyl group having 1 to 3 carbon atoms.

In the general formula (I), the -A-Y group is preferably a formyl group, a fluorosulfonyl group, an alkylsulfonyl group having 1 to 4 carbon atoms, in which at least one hydrogen atom may be substituted with a halogen atom, an alkenylsulfonyl group having 2 to 4 carbon atoms, in which at least one hydrogen atom may be substituted with a halogen atom, an arylsulfonyl group having 6 to 10 carbon atoms, in which at least one hydrogen atom may be substituted with a halogen atom, an alkylcarbonyl group having 1 to 4 carbon atoms, in which at least one hydrogen atom may be substituted with a halogen atom, an alkenylcarbonyl group having 2 to 6 carbon atoms, an alkynylcarbonyl group having 3 to 6 carbon atoms, an arylcarbonyl group having 6 to 10 carbon atoms, in which at least one hydrogen atom may be substituted with a halogen atom, an alkoxycarbonyl group having 2 to 5 carbon atoms, in which at least one hydrogen atom may be substituted with a halogen atom, an alkenyloxycarbonyl group having 3 to 5 carbon atoms, an alkynyloxycarbonyl group having 4 to 6 carbon atoms, an aryloxycarbonyl group having 7 to 10 carbon atoms, in which at least one hydrogen atom may be substituted with a halogen atom, a —C(═O)-L¹-C(═O)OR¹ group, a —C(═O)-L²-P(═O)(OR)(OR²) group, or a —P(═O)(OR)(OR²) group, and preferably a fluorosulfonyl group, an alkylsulfonyl having 1 to 2 carbon atoms, an alkenylsulfonyl group having 2 to 3 carbon atoms, an arylsulfonyl group having 6 to 8 carbon atoms, a formyl group, an alkylcarbonyl group having 1 to 2 carbon atoms, an alkenylcarbonyl group having 2 to 4 carbon atoms, an arylcarbonyl group having 7 to 9 carbon atoms, an alkoxycarbonyl group having 2 to 3 carbon atoms, an alkenyloxycarbonyl group having 3 to 4 carbon atoms, an alkynyloxycarbonyl group having 4 to 5 carbon atoms, an aryloxycarbonyl group having 7 to 9 carbon atoms, in which at least one hydrogen atom may be substituted with a halogen atom, a —C(═O)-L¹-C(═O)OR¹ group, a —C(═O)-L²-P(═O)(OR)(OR²) group, or a —P(═O)(OR)(OR²) group.

As specific examples of the -A-Y group in the foregoing general formula (I), there are exemplified the following (i) to (xvii) group and the like.

(i) Linear alkanesulfonyl groups, such as a fluorosulfonyl group, a methanesulfonyl group, an ethanesulfonyl group, a propane-1-sulfonyl group, a butane-1-sulfonyl group, a pentane-1-sulfonyl group, a hexane-1-sulfonyl group, etc.
(ii) Branched alkanesulfonyl groups, such as a propane-2-sulfonyl group, a butane-2-sulfonyl group, a 2-methylpropane-2-sulfonyl group, a 2-methylbutane-2-sulfonyl group, etc.
(iii) Alkenylsulfonyl groups, such as a vinylsulfonyl group, a 2-propene-1-sulfonyl group, a 2-propene-2-sulfonyl group, etc.
(iv) Alkanesulfonyl groups in which a part of hydrogen atoms is substituted with a fluorine atom, such as a fluoromethanesulfonyl group, a trifluoromethanesulfonyl group, a 2,2,2-trifluoroethanesulfonyl group, etc.
(v) Arylsulfonyl groups, such as a benzenesulfonyl group, a 2-methylbenzenesulfonyl group, a 3-methylbenzenesulfonyl group, a 4-methylbenzene sulfonyl group, a 4-tert-butylbenzenesulfonyl group, a 2,4,6-trimethylbenzenesulfonyl group, a 2-fluorobenzenesulfonyl group, a 3-fluorobenzenesulfonyl group, a 4-fluorobenzenesulfonyl group, a 2,4-difluorobenzenesulfonyl group, a 2,6-difluorobenzenesulfonyl group, a 3,4-difluorobenzenesulfonyl group, a 2,4,6-trifluorobenzenesulfonyl group, a pentafluorobenzenesulfonyl group, a 4-(trifluoromethyl)benzenesulfonyl group, etc.
(vi) Linear alkylcarbonyl groups, such as a methylcarbonyl group, an ethylcarbonyl group, an n-propylcarbonyl group, an n-butylcarbonyl group, an n-pentylcarbonyl group, an n-hexylcarbonyl group, etc.
(vii) Branched alkoxycarbonyl groups, such as an isopropylcarbonyl group, a sec-butylcarbonyl group, a tert-butylcarbonyl group, a tert-amylcarbonyl group, etc.
(viii) Alkoxycarbonyl groups in which a part of hydrogen atoms is substituted with a fluorine atom, such as a fluoromethylcarbonyl group, a trifluoromethylcarbonyl group, a 2,2,2-trifluoroethylcarbonyl group, etc.
(ix) Alkenylcarbonyl groups, such as a vinylcarbonyl group, a 1-propenylcarbonyl group, a 2-propenylcarbonyl group, a 1-methyl-2-propenylcarbonyl group, a 1,1-dimethyl-2-propenylcarbonyl group, a 1-butenylcarbonyl group, a 2-butenylcarbonyl group, a 3-butenylcarbonyl group, a 2-pentenylcarbonyl group, a 2-hexenylcarbonyl group, etc.
(x) Alkynylcarbonyl groups, such as a 2-propynylcarbonyl group, a 2-butynylcarbonyl group, a 3-butynylcarbonyl group, a 4-pentynylcarbonyl group, a 5-hexynylcarbonyl group, a 1-methyl-2-propynylcarbonyl group, a 1-methyl-2-butynylcarbonyl group, a 1,1-dimethyl-2-propynylcarbonyl group, etc.
(xi) Arylcarbonyl groups, such as a phenylcarbonyl group, a 2-methylphenylcarbonyl group, a 3-methylphenylcarbonyl group, a 4-methylphenylcarbonyl group, a 4-tert-butylphenylcarbonyl group, a 2,4,6-trimethylphenylcarbonyl group, a 2-fluorophenylcarbonyl group, a 3-fluorophenylcarbonyl group, a 4-fluorophenylcarbonyl group, a 2,4-difluorophenylcarbonyl group, a 2,6-difluorophenylcarbonyl group, a 3,4-difluorophenylcarbonyl group, a 2,4,6-trifluorophenylcarbonyl group, a pentafluorophenylcarbonyl group, a 2-(trifluoromethyl)phenylcarbonyl group, a 3-(trifluoromethyl)phenylcarbonyl group, etc.
(xii) Linear alkoxycarbonyl groups, such as a methoxycarbonyl group, an ethoxycarbonyl group, an n-propoxycarbonyl group, an n-butoxycarbonyl group, an n-pentyloxycarbonyl group, an n-hexyloxycarbonyl group, etc.
(xiii) Branched alkoxycarbonyl groups, such as an isopropoxycarbonyl group, a sec-butoxycarbonyl group, a tert-butoxycarbonyl group, a tert-amyloxycarbonyl group, etc.
(xiv) Alkoxycarbonyl groups in which a part of hydrogen atoms is substituted with a fluorine atom, such as a fluoromethoxycarbonyl group, a trifluoromethoxycarbonyl group, a 2,2,2-trifluoroethoxycarbonyl group, etc.
(xv) Alkenyloxycarbonyl groups, such as a vinyloxycarbonyl group, a 1-propenyloxycarbonyl group, a 2-propenyloxycarbonyl group, a 1-methyl-2-propenyloxycabonyl group, a 1,1-dimethyl-2-propenyloxycarbonyl group, a 1-butenyloxycarbonyl group, a 2-butenyloxycarbonyl group, a 3-butenyloxycarbonyl group, a 2-pentenyloxycarbonyl group, a 2-hexenyloxycarbonyl group, etc.
(xvi) Alkynyloxycarbonyl groups, such as a 2-propynyloxycarbonyl group, a 2-butynyloxycarbonyl group, a 3-butynyloxycarbonyl group, a 4-pentynyloxycarbonyl group, a 5-hexynyloxycarbonyl group, a 1-methyl-2-propynyloxycarbonyl group, a 1-methyl-2-butynyloxycarbonyl group, a 1,1-dimethyl-2-propynyloxycarbonyl group, etc.
(xvii) Aryloxycarbonyl groups, such as a phenyloxycarbonyl group, a 2-methylphenyloxycarbonyl group, a 3-methylphenyloxycarbonyl group, a 4-methylphenyloxycarbonyl group, a 4-tert-butylphenyloxycarbonyl group, a 2,4,6-trimethylphenyloxycarbonyl group, a 2-fluorophenyloxycarbonyl group, a 3-fluorophenyloxycarbonyl group, a 4-fluorophenyloxycarbonyl group, a 2,4-difluorophenyloxycarbonyl group, a 2,6-difluorophenyloxycarbonyl group, a 3,4-difluorophenyloxycarbonyl group, a 2,4,6-trifluorophenyloxycarbonyl group, a pentafluorophenyloxycarbonyl group, a 2-(trifluoromethyl)phenyloxycarbonyl group, a 3-trifluoro methylphenyloxycarbonyl group, a 4-(trifluoromethyl)phenyloxycarbonyl group, a 4-fluoro-3-(trifluoromethyl)phenyloxycarbonyl group, a 4-chloro-3-(trifluoromethyl)phenyloxycarbonyl group, etc.

Among the aforementioned the -A-Y groups, a methanesulfonyl group, an ethanesulfonyl group, a propanesulfonyl group, a butanesulfonyl group, a vinylsulfonyl group, a 2-propene-1-sulfonyl group, a benzenesulfonyl group, a 2-methylbenzenesulfonyl group, a 3-methylbenzenesulfonyl group, a 4-methylbenzenesulfonyl group, a methylcarbonyl group, an ethylcarbonyl group, an n-propylcarbonyl group, a vinylcarbonyl group, a 2-propynylcarbonyl group, a 2-butynylcarbonyl group, a 3-butynylcarbonyl group, a phenylcarbonyl group, a 2-methylphenylcarbonyl group, a 3-methylphenylcarbonyl group, a 4-methylphenylcarbonyl group, a 2-trifluoromethylphenylcarbonyl group, a 3-(trifluoromethyl)phenylcarbonyl group, a 4-(trifluoromethyl)phenylcarbonyl group, a methoxycarbonyl group, an ethoxycarbonyl group, an n-propoxycarbonyl group, a 2-propynyloxycarbonyl group, a 2-butynyloxycarbonyl group, a 3-butynyloxycarbonyl group, a phenyloxycarbonyl group, a 2-methylphenyloxycarbonyl group, a 3-methylphenyloxycarbonyl group, a 4-methylphenyloxycarbonyl group, a 2-(trifluoromethyl)phenyloxycarbonyl group, a 3-(trifluoromethyl)phenyloxycarbonyl group, a 4-(trifluoromethyl)phenyloxycarbonyl group, a 4-chloro-3-(trifluoromethyl)phenyloxycarbonyl group, a 4-fluoro-3-(trifluoromethyl)phenyloxycarbonyl group, and one or more groups represented by the following formulae, are preferred.

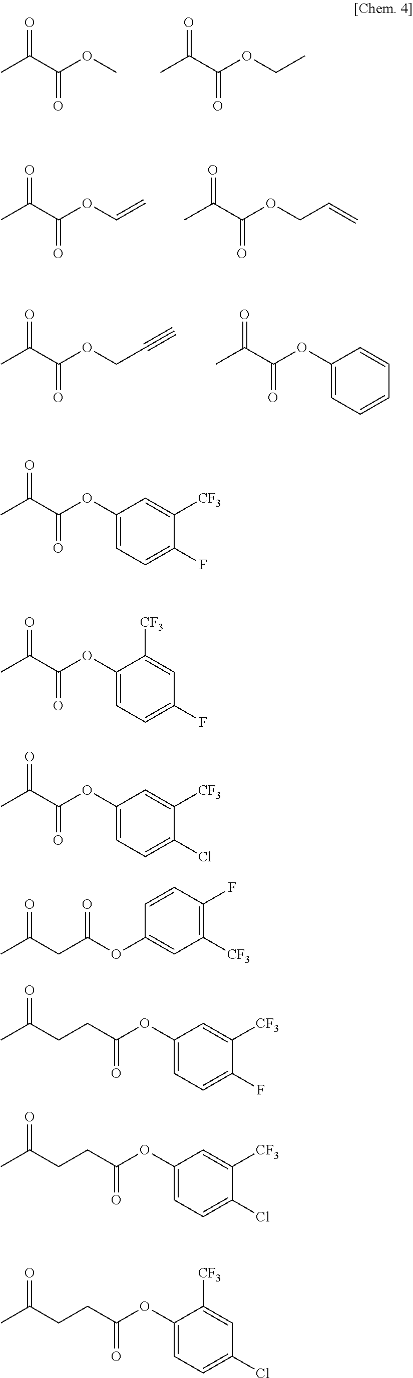

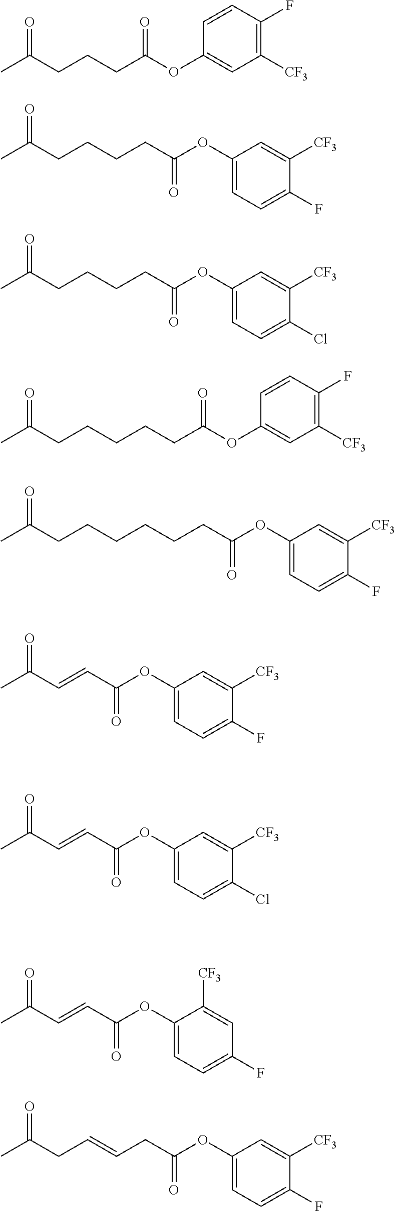

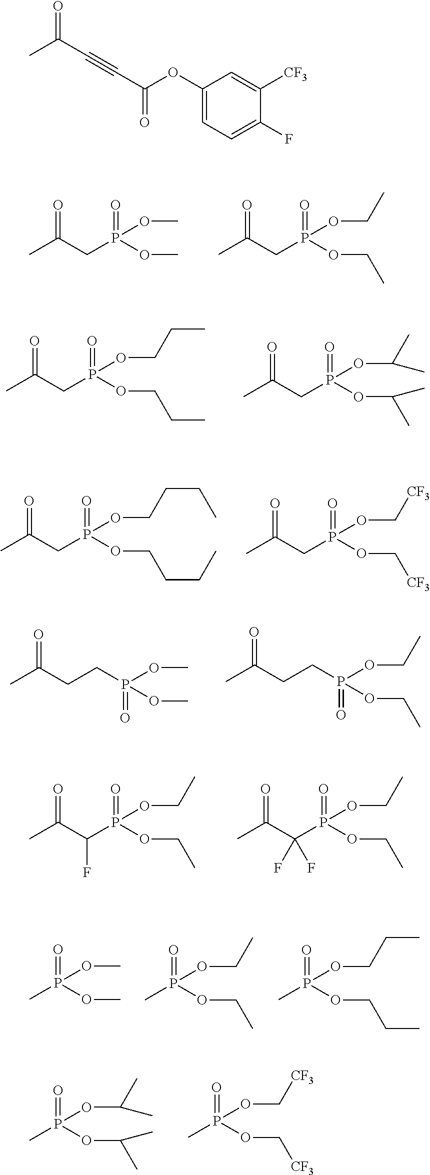

As more preferred specific examples of the -A-Y group, there are exemplified a methanesulfonyl group, an ethanesulfonyl group, a methoxycarbonyl group, an ethoxycarbonyl group, an n-propoxycarbonyl group, a 2-propynyloxycarbonyl group, a 2-butynyloxycarbonyl group, a 3-butynyloxycarbonyl group, a phenyloxycarbonyl group, a 2-methylphenyloxycarbonyl group, a 3-methylphenyloxycarbonyl group, a 4-methylphenyloxycarbonyl group, a 2-(trifluoromethyl)phenyloxycarbonyl group, a 3-(trifluoromethyl)phenyloxycarbonyl group, a 4-(trifluoromethyl)phenyloxycarbonyl group, a 4-chloro-3-(trifluoromethyl)phenyloxycarbonyl group, a 4-fluoro-3-(trifluoromethyl)phenyloxycarbonyl group, and one or more groups represented by the following formulae.

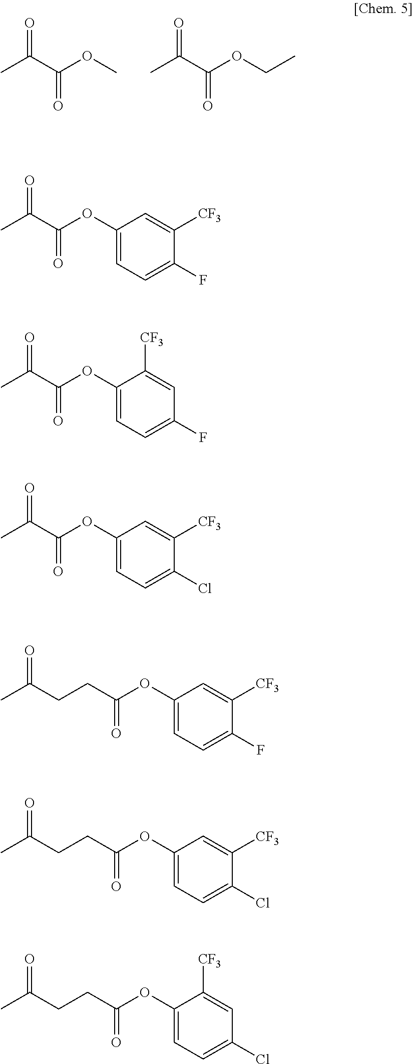

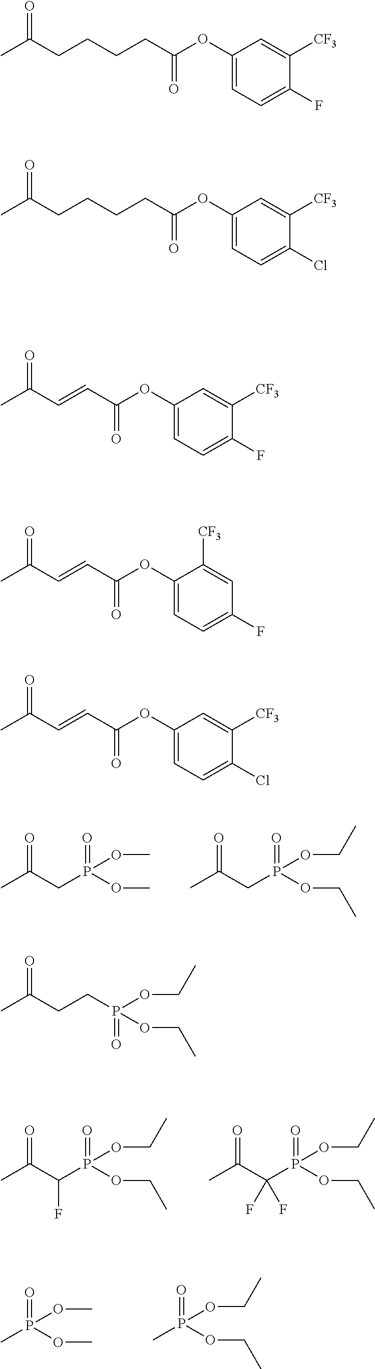

The case of the aforementioned range of substituents is preferred because the electrochemical characteristics over a wide temperature range may be significantly improved.

The effect for improving the electrochemical characteristics over a wide temperature range also relies on the substitution position of R_{f} or X on the benzene ring, and those having R_{f} at at least one of the para position and the meta position are preferred, and those having X at at least one of the ortho position and the para position are preferred. Those having R_{f} at the meta position are especially preferred.

Specifically, examples of the compound represented by the foregoing general formula (I) include compounds represented by the following formulae.

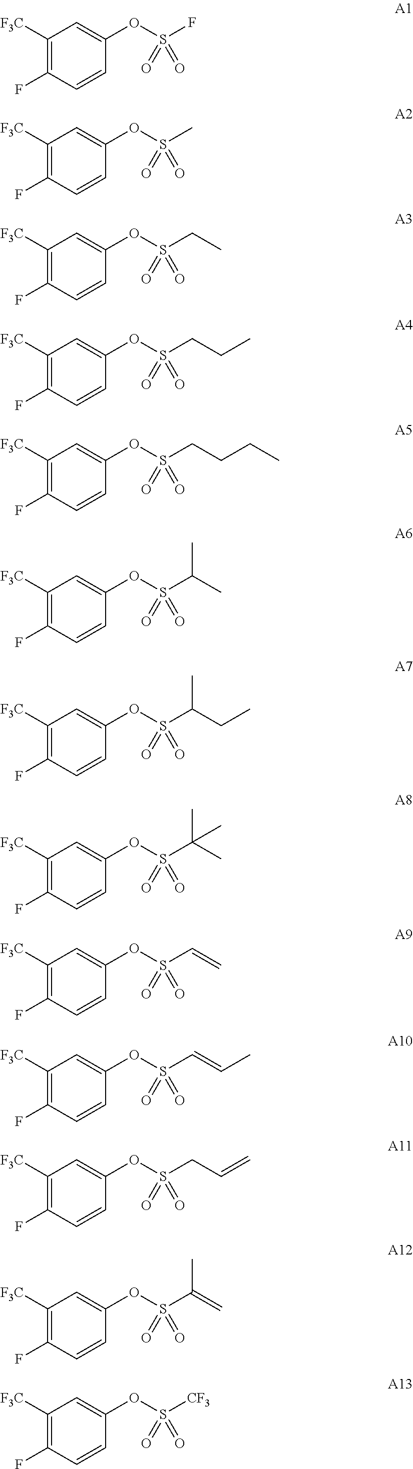

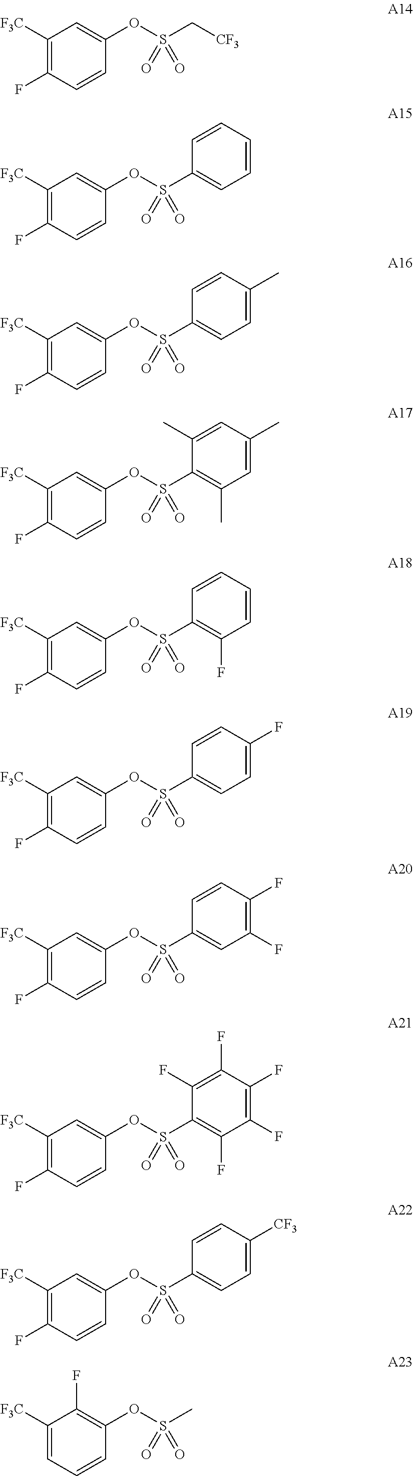

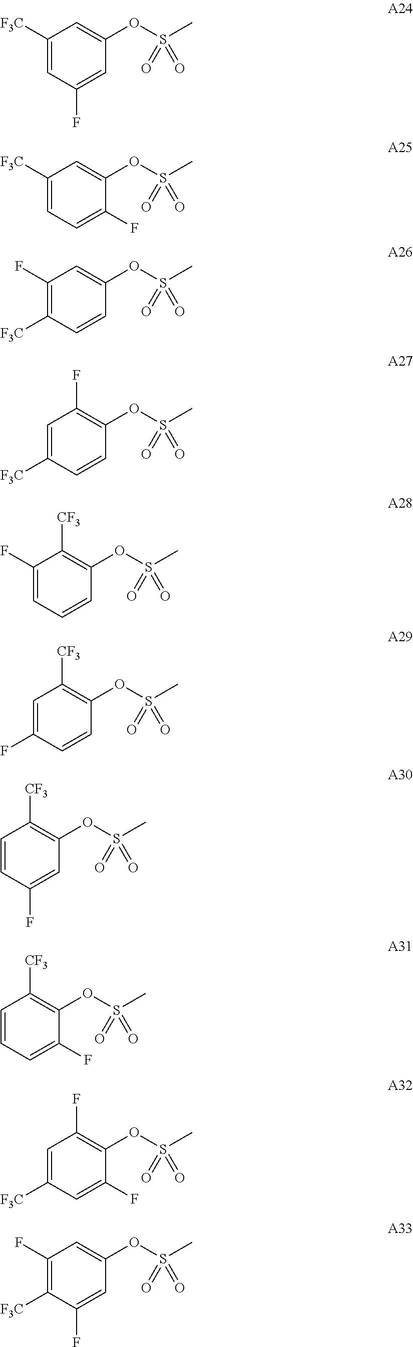

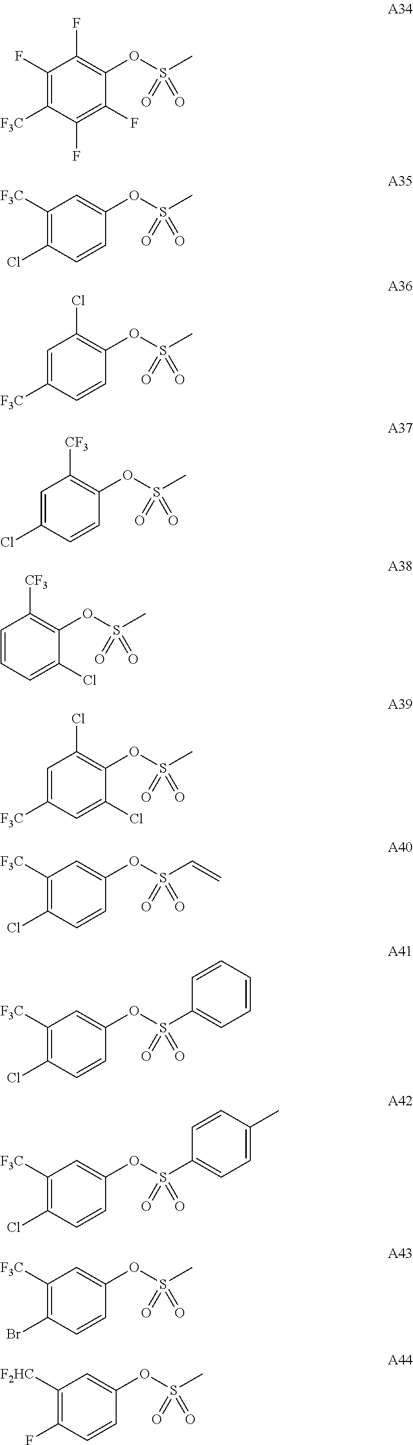

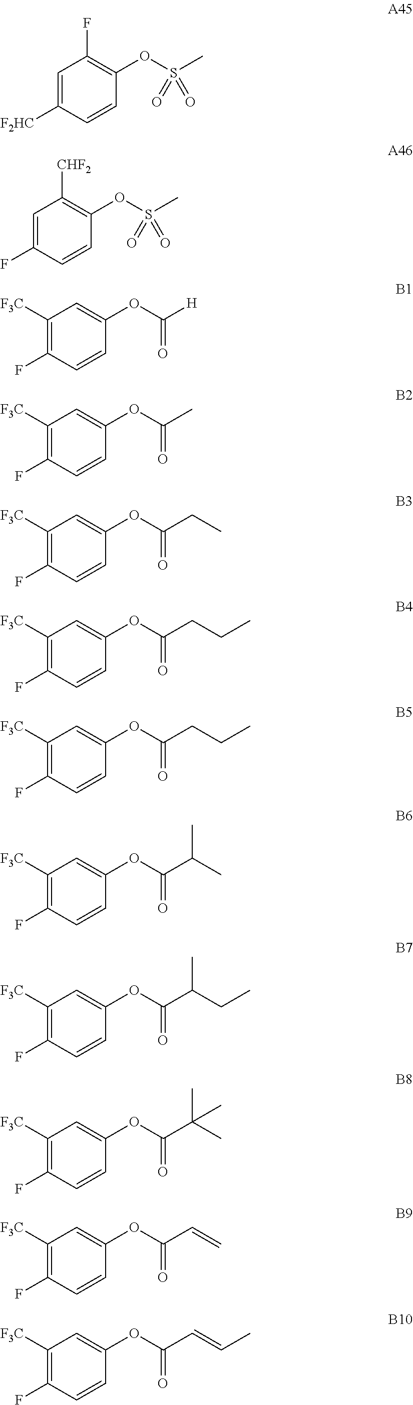

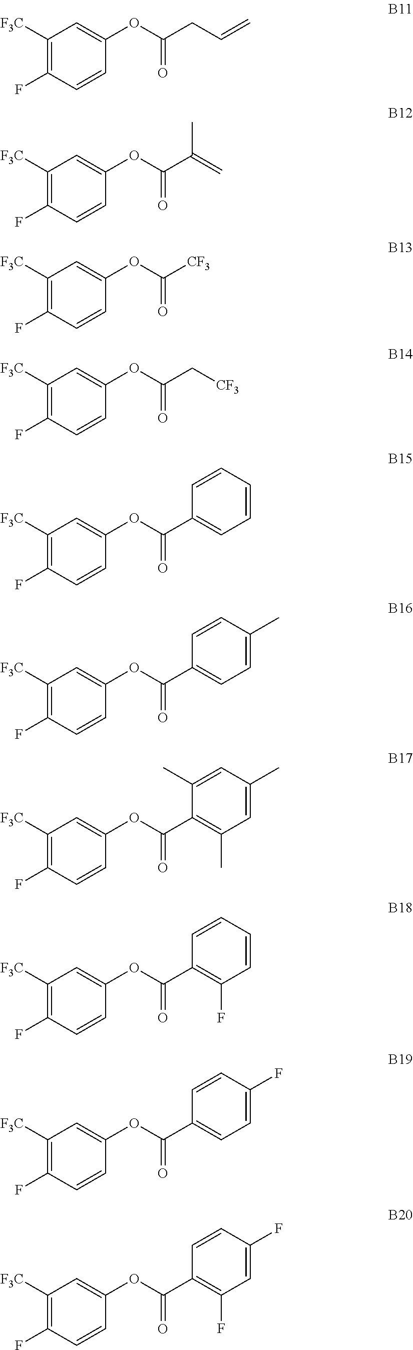

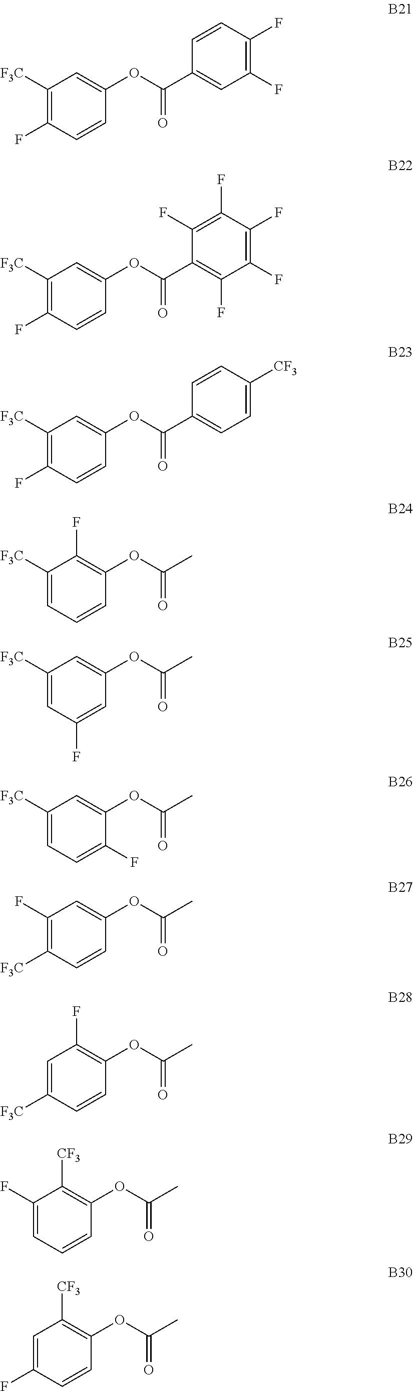

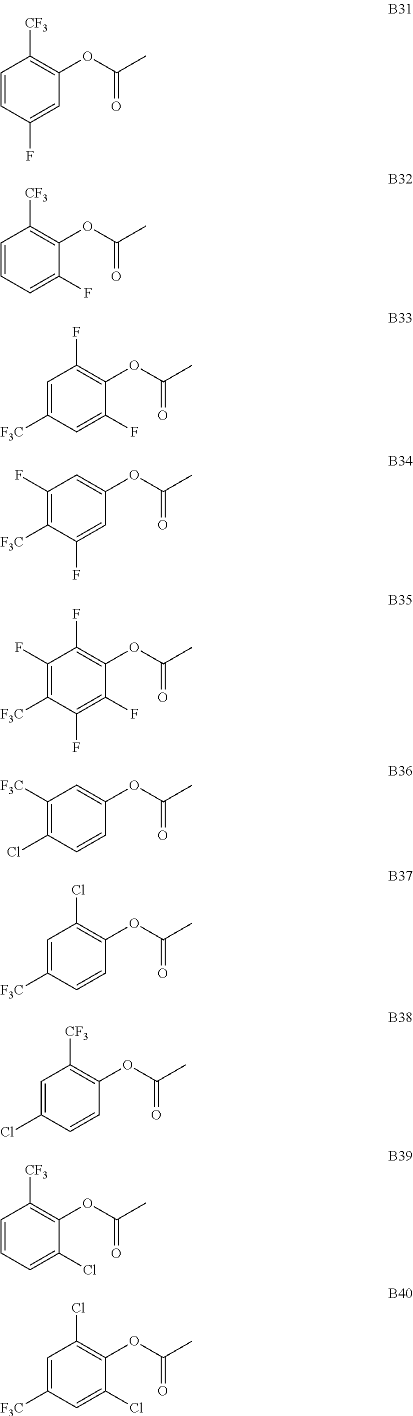

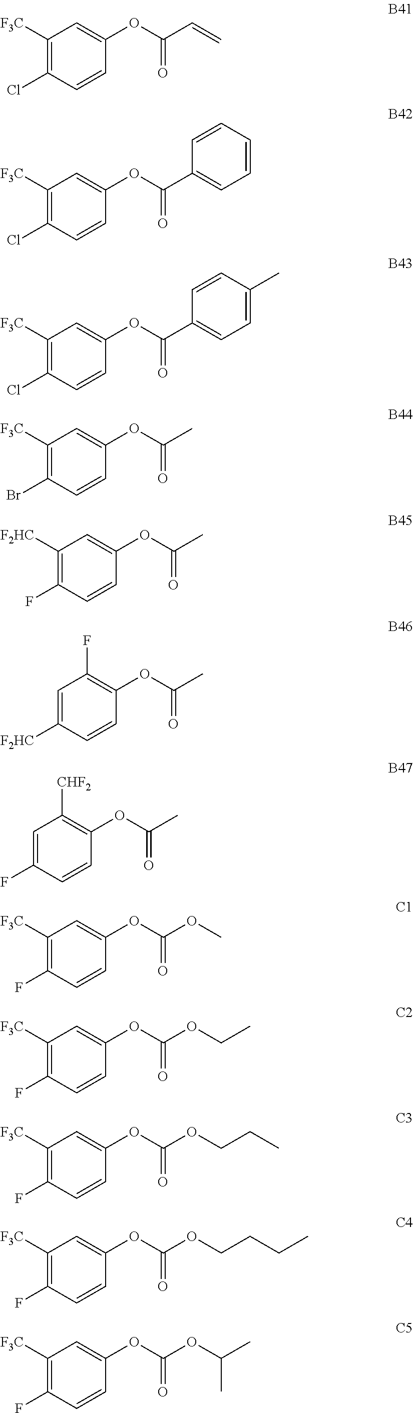

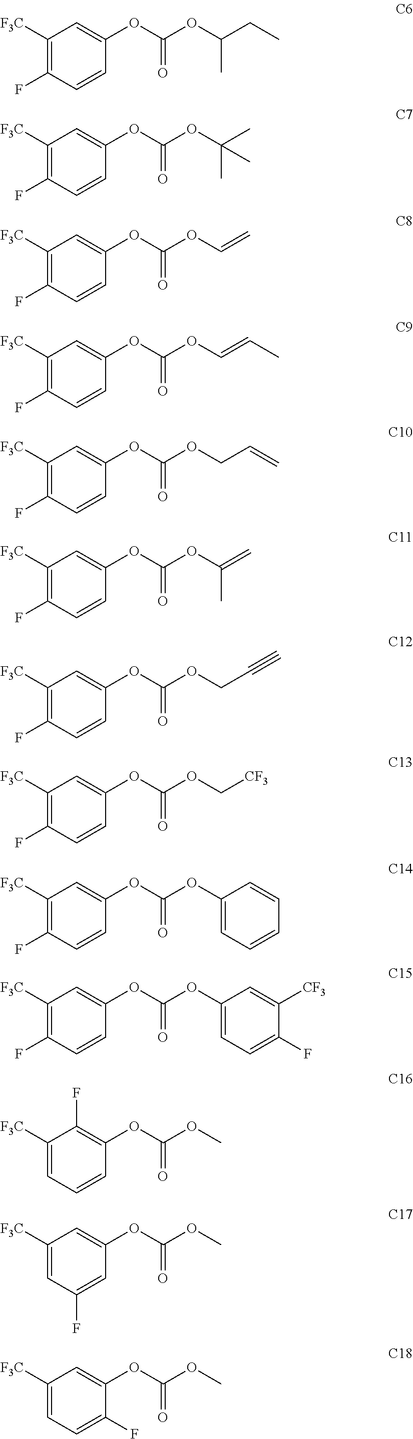

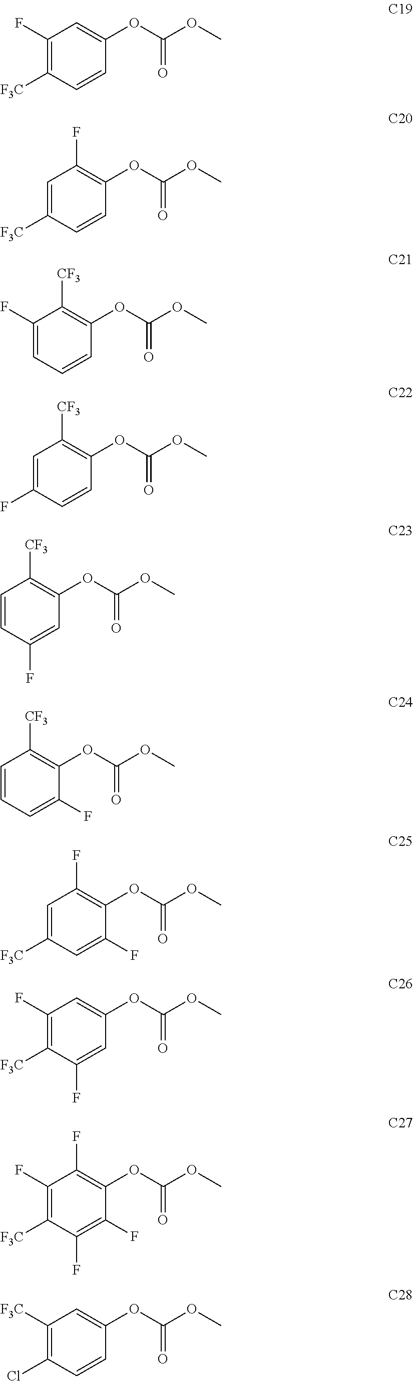

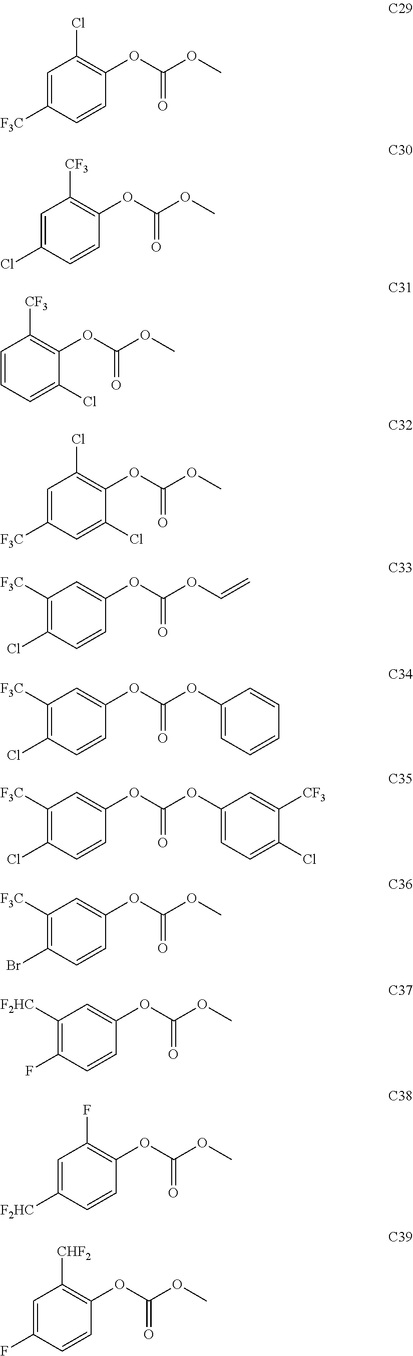

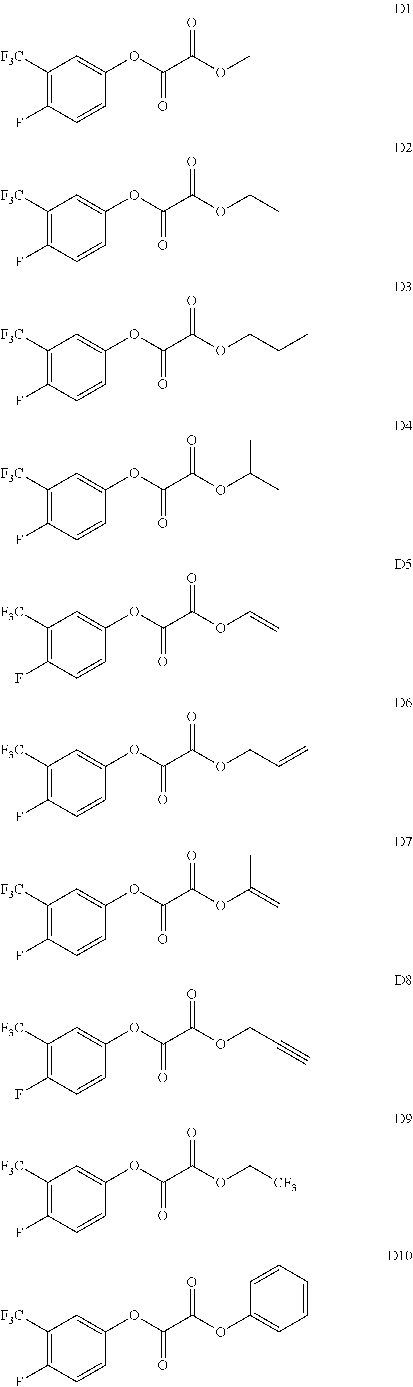

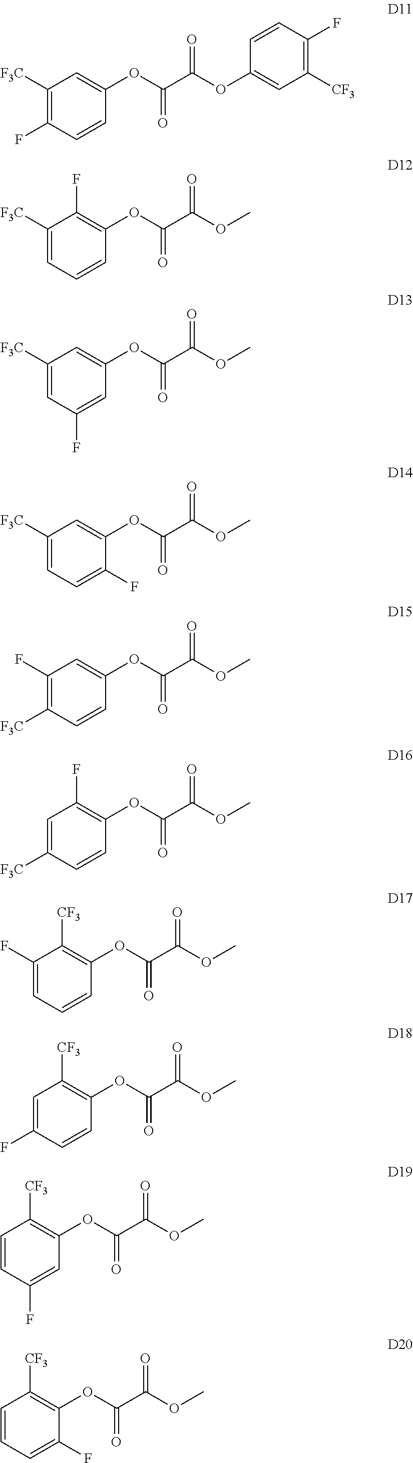

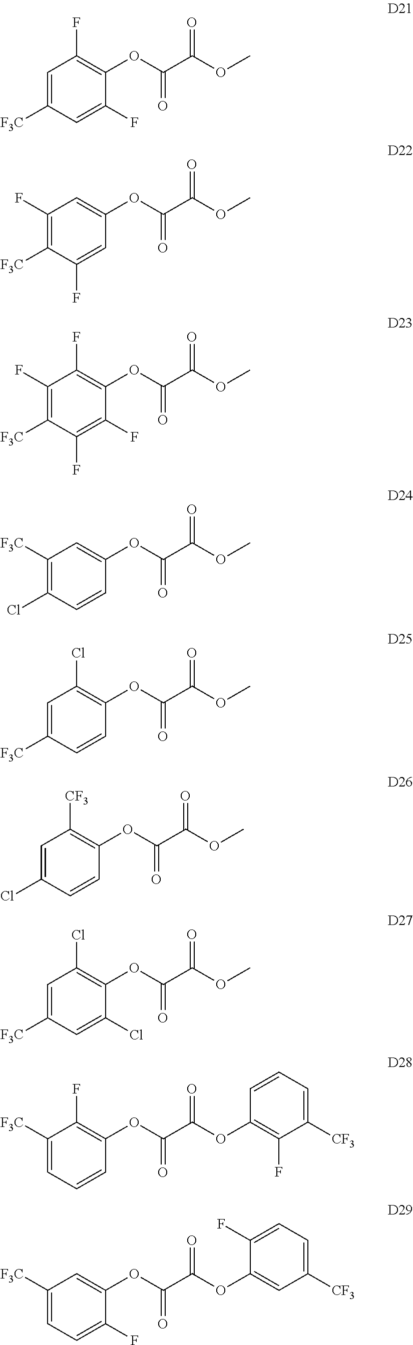

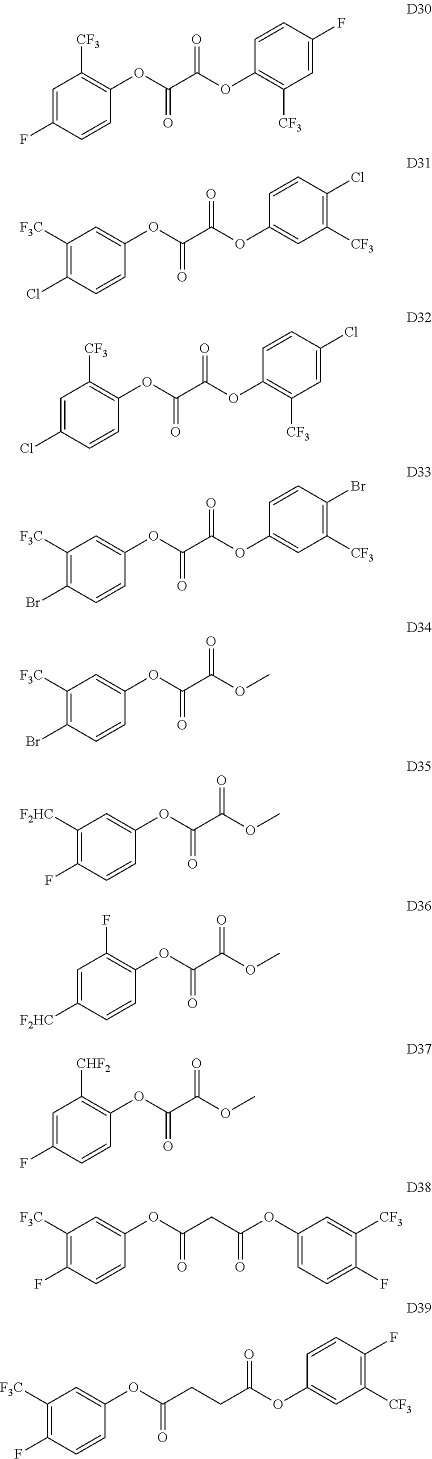

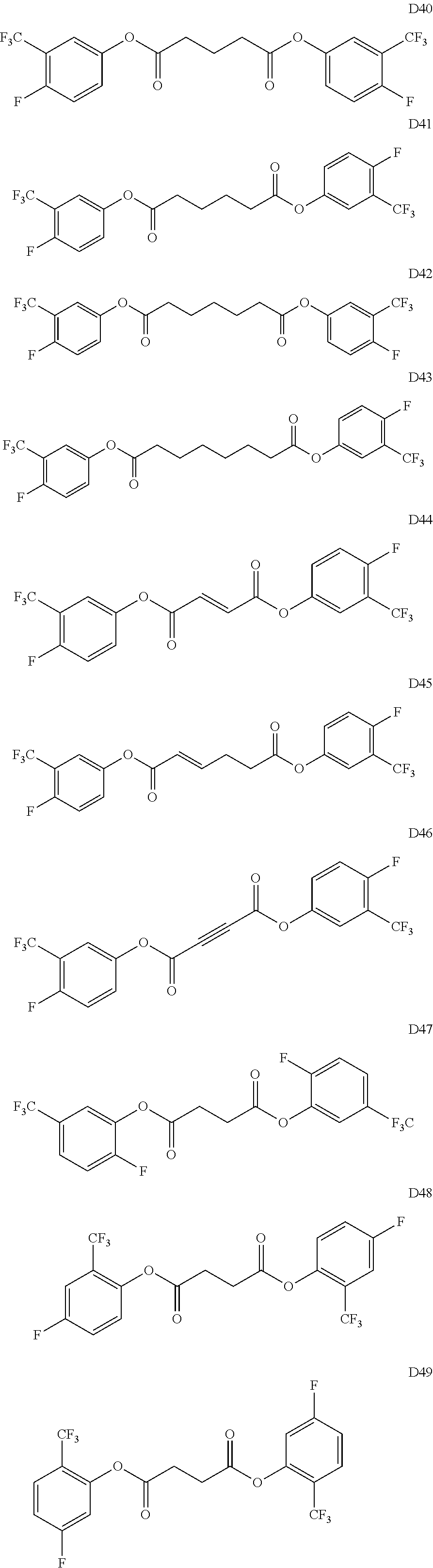

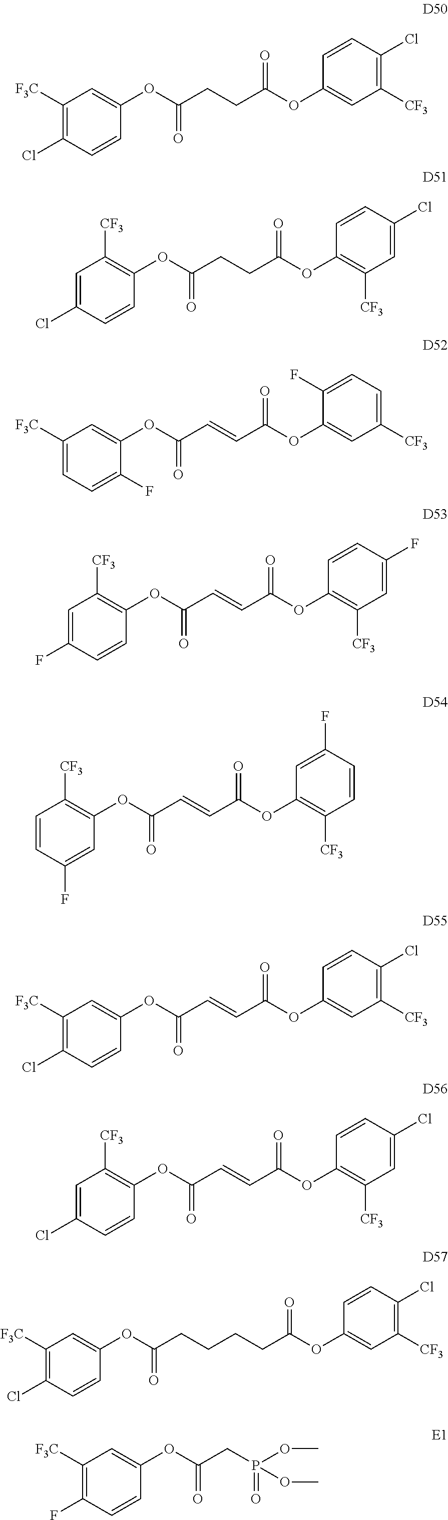

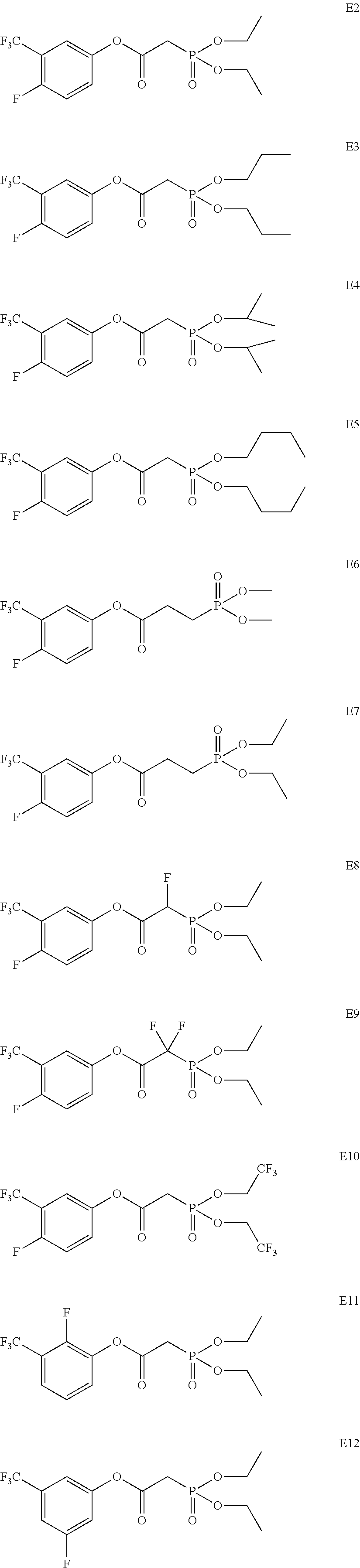

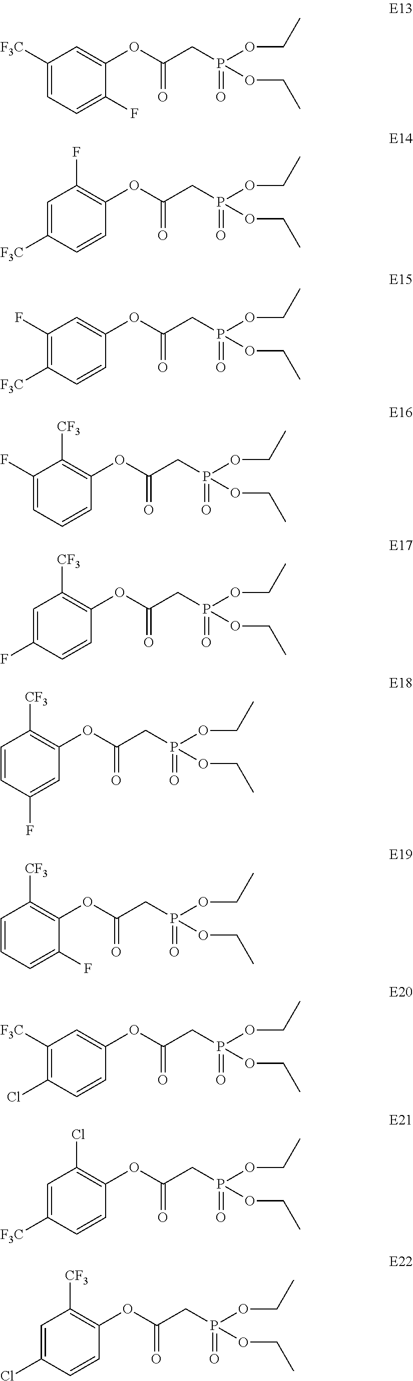

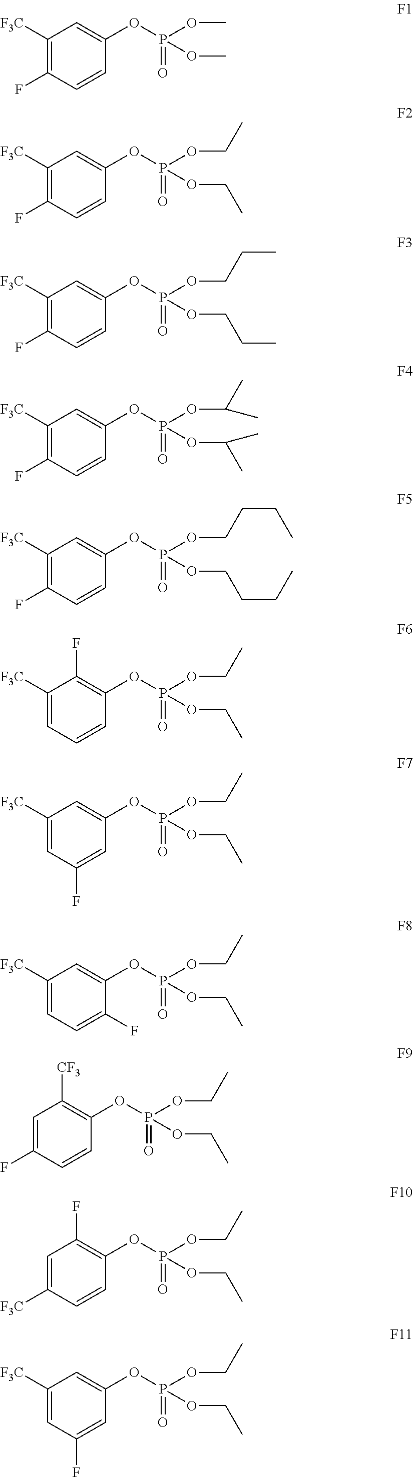

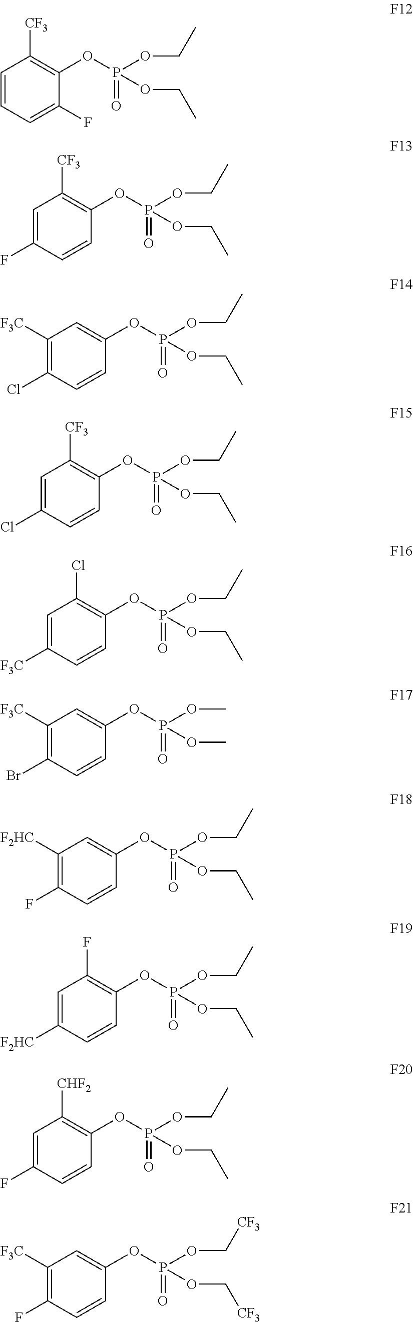

Among the aforementioned compounds, the structural formulae A1 to A4, A6, A9 to A11, A13, A15, A16, A23 to A33, A35 to A43, B1 to B4, B8 to B13, B15, B24 to B34, B36 to B42, B44, C1 to C3, C8 to C12, C15 to C26, C28 to C33, C35, C36, D1 to D3, D5 to D8, D11 to D22, D24 to D34, D36 to D42, D44 to D57, E1 to E4, E8 to E22, F1 to F4, F6 to F16, and F21 are preferred; and the structural formulae A2, A3, A6, A9, A15, A16, A25, A29, A35, A36, A40 to A42, B1 to B3, B9, B12, B26, B30, B36 to B38, B41, C1, C2, C8, C10, C12, C15, C22, C28 to C30, C33, C35, D1, D2, D5, D6, D8, D11, D18, D24 to D26, D30 to D32, D39, D41, D44, D48, D50, D51, D53, D55 to D57, E1, E2, E8, E10, E14, E17, E20 to E22, F1, F2, F9, F10, and F14 to F16 are more preferred.

Among the compounds represented by the foregoing general formula (I), as still more preferred specific examples, there are exemplified 4-fluoro-3-(trifluoromethyl)phenyl methanesulfonate (structural formula A2), 4-fluoro-3-(trifluoromethyl)phenyl propane-2-sulfonate (structural formula A6), 4-fluoro-3-(trifluoromethyl)phenyl vinylsulfonate (structural formula A9), 4-fluoro-3-(trifluoromethyl)phenyl 4-methylbenzenesulfonate (structural formula A16), 2-fluoro-3-(trifluoromethyl)phenyl methanesulfonate (structural formula A25), 4-fluoro-2-(trifluoromethyl)phenyl methane sulfonate (structural formula A29), 3-chloro-4-(trifluoromethyl)phenyl methanesulfonate (structural formula A35), 4-chloro-3-(trifluoromethyl)phenyl methanesulfonate (structural formula A36), 4-chloro-3-(trifluoromethyl)phenyl vinylsulfonate (structural formula A40), 4-chloro-3-(trifluoromethyl)phenyl 4-methylbenzenesulfonate (structural formula A42), 4-fluoro-3-(trifluoromethyl)phenyl acetate (structural formula B2), 4-fluoro-3-(trifluoromethyl)phenyl acrylate (structural formula B9), 4-fluoro-3-(trifluoromethyl)phenyl methacrylate (structural formula B12), 4-chloro-3-(trifluoromethyl)phenyl acetate (structural formula B36), 4-chloro-3-(trifluoromethyl)phenyl acrylate (structural formula B41), 4-fluoro-3-(trifluoromethyl)phenyl methyl carbonate (structural formula C1), bis(4-fluoro-3-(trifluoromethyl)phenyl)carbonate (structural formula C15), 4-chloro-3-(trifluoromethyl)phenyl methyl carbonate (structural formula C28), 4-chloro-3-(trifluoromethyl)phenyl vinyl carbonate (structural formula C33), bis(4-chloro-3-(trifluoromethyl)phenyl)carbonate (structural formula C35), 4-fluoro-3-(trifluoromethyl)phenyl methyl oxalate (structural formula D1), bis(4-fluoro-3-(trifluoromethyl)phenyl) oxalate (structural formula D11), 4-chloro-3-(trifluoromethyl)phenyl methyl oxalate (structural formula D24), bis(4-chloro-3-(trifluoromethyl)phenyl) oxalate (structural formula D31), bis(4-fluoro-3-(trifluoromethyl)phenyl) succinate (structural formula D39), bis(4-fluoro-3-(trifluoromethyl)phenyl) adipate (structural formula D41), bis(4-fluoro-3-(trifluoromethyl)phenyl)fumarate (structural formula D44), bis(4-chloro-3-(trifluoromethyl)phenyl) succinate (structural formula D50), bis(4-chloro-3-(trifluoromethyl)phenyl)fumarate (structural formula D55), bis(4-chloro-3-(trifluoromethyl)phenyl)adipate (structural formula D57), 4-fluoro-3-(trifluoromethyl)phenyl 2-(dimethoxyphosphoryl)acetate (structural formula E1), 4-fluoro-3-(trifluoromethyl)phenyl 2-(diethoxyphosphoryl)acetate (structural formula E2), 4-fluoro-3-(trifluoromethyl)phenyl 2-(diethoxyphosphoryl)-2-fluoroacetate (structural formula E8), 4-chloro-3-(trifluoromethyl)phenyl 2-(diethoxyphosphoryl)acetate (structural formula E20), 4-fluoro-3-(trifluoromethyl)phenyl dimethylphosphate (structural formula F1), 4-fluoro-3-(trifluoro methyl)phenyl diethylphosphate (structural formula F2), and 4-chloro-3-(trifluoro methyl)phenyl diethylphosphate (structural formula F14).

Among these suitable examples, one or more selected from 4-fluoro-3-(trifluoromethyl)phenyl methanesulfonate (structural formula A2), 4-fluoro-3-(trifluoromethyl)phenyl propane-2-sulfonate (structural formula A6), 4-fluoro-3-(trifluoromethyl)phenyl vinylsulfonate (structural formula A9), 4-fluoro-3-(trifluoro methyl)phenyl 4-methylbenzenesulfonate (structural formula A16), 2-fluoro-3-(trifluoromethyl)phenyl methane sulfonate (structural formula A25), 4-fluoro-2-(trifluoromethyl)phenyl methanesulfonate (structural formula A29), 3-chloro-4-(trifluoromethyl)phenyl methanesulfonate (structural formula A35), 4-chloro-3-(trifluoromethyl)phenyl methanesulfonate (structural formula A36), 4-fluoro-3-(trifluoromethyl)phenyl acetate (structural formula B2), 4-fluoro-3-(trifluoro methyl)phenyl methacrylate (structural formula B12), 4-chloro-3-(trifluoromethyl)phenyl acrylate (structural formula B41), 4-fluoro-3-(trifluoromethyl)phenyl methyl carbonate (structural formula C1), bis(4-fluoro-3-(trifluoromethyl)phenyl carbonate (structural formula C15), 4-chloro-3-(trifluoromethyl)phenyl vinyl carbonate (structural formula C33), 4-fluoro-3-(trifluoromethyl)phenyl methyl oxalate (structural formula D1), bis(4-fluoro-3-(trifluoromethyl)phenyl) oxalate (structural formula D11), bis(4-fluoro-3-(trifluoromethyl)phenyl) succinate (structural formula D39), bis(4-fluoro-3-(trifluoromethyl)phenyl)fumarate (structural formula D44), bis(4-chloro-3-(trifluoromethyl)phenyl) adipate (structural formula D57), 4-fluoro-3-(trifluoromethyl)phenyl 2-(diethoxyphosphoryl)acetate (structural formula E2), and 4-fluoro-3-(trifluoromethyl)phenyl diethylphosphate (structural formula F2) are especially preferred.

In the nonaqueous electrolytic solution of the present invention, a content of the phenyl ester compound represented by the general formula (I), in which the benzene ring is substituted with both a halogen atom and a fluoroalkyl group, is preferably 0.001 to 5% by mass in the nonaqueous electrolytic solution. When the content is 5% by mass or less, there is less concern that in the case where a battery in which a surface film is excessively formed on the electrode is used at a high temperature, the cycle property is worsened, and when it is 0.001% by mass or more, a surface film is sufficiently formed, and an effect for improving the cycle property in the case of using the battery at a high voltage is increased. The content is preferably 0.01% by mass or more, and more preferably 0.1% by mass or more in the nonaqueous electrolytic solution. In addition, an upper limit thereof is preferably 4% by mass or less, and more preferably 2% by mass or less.

In the nonaqueous electrolytic solution of the present invention, by combining the phenyl ester compound represented by the general formula (I), in which the benzene ring is substituted with both a halogen atom and a fluoroalkyl group, with a nonaqueous solvent and an electrolyte salt as described below, a peculiar effect such that not only the capacity retention rate after a cycle in the case of using the energy storage device at a high temperature may be improved, but also the gas generation may be inhibited is revealed.

### [Nonaqueous Solvent]

Examples of the nonaqueous solvent which is used for the nonaqueous electrolytic solution of the present invention include cyclic carbonates, linear esters, lactones, ethers, and amides; and it is preferred that the nonaqueous solvent includes both a cyclic carbonate and a linear ester.

It is to be noted that the term, linear ester, is used as a concept including a linear carbonate and a linear carboxylic acid ester.

As the cyclic carbonate, one or more selected from ethylene carbonate (EC), propylene carbonate (PC), 1,2-butylene carbonate, 2,3-butylene carbonate, a cyclic carbonate having a fluorine atom or an unsaturated bond, and the like are exemplified; and one or more selected from EC, PC, and a cyclic carbonate having a fluorine atom or an unsaturated bond are preferred.

As the cyclic carbonate having a fluorine atom, one or more selected from 4-fluoro-1,3-dioxolan-2-one (FEC) and trans- or cis-4,5-difluoro-1,3-dioxolan-2-one (the both will be hereunder named generically as “DFEC”) are preferred; and FEC is more preferred.

As the cyclic carbonate having an unsaturated bond, such as a carbon-carbon double bond, a carbon-carbon triple bond, etc., one or more selected from vinylene carbonate (VC), vinyl ethylene carbonate (VEC), 4-ethynyl-1,3-dioxolan-2-one (EEC), and the like are exemplified; and one or more selected from VC, VEC, and EEC are preferred.

Use of at least one of the aforementioned cyclic carbonates having a fluorine atom or an unsaturated bond is preferred because the gas generation after a cycle in the case of using the energy storage device at a high temperature may be much more inhibited; and it is more preferred to include both the cyclic carbonate containing a fluorine atom and the cyclic carbonate having an unsaturated bond as described above.

A content of the aforementioned cyclic carbonate having an unsaturated bond is preferably 0.07% by volume or more, more preferably 0.2% by volume or more, and still more preferably 0.7% by volume or more relative to a total volume of the nonaqueous solvent; and when an upper limit thereof is preferably 7% by volume or less, more preferably 4% by volume or less, and still more preferably 2.5% by volume or less, stability of a surface film is increased, and the cycle property in the case of using the energy storage device at a high temperature is improved, and hence, such is preferred.

A content of the cyclic carbonate having a fluorine atom is preferably 0.07% by volume or more, more preferably 4% by volume or more, and still more preferably 7% by volume or more relative to a total volume of the nonaqueous solvent; and when an upper limit thereof is preferably 35% by volume or less, more preferably 25% by volume or less, and still more preferably 15% by volume or less, stability of a surface film is increased, and the cycle property in the case of using the energy storage device at a high temperature is improved, and hence, such is preferred.

In the case where the nonaqueous solvent includes both the cyclic carbonate having an unsaturated bond and the cyclic carbonate having a fluorine atom as described above, a proportion of the content of the cyclic carbonate having an unsaturated bond to the content of the cyclic carbonate having a fluorine atom is preferably 0.2% or more, more preferably 3% or more, and still more preferably 7% or more; and when an upper limit thereof is preferably 40% or less, more preferably 30% or less, and still more preferably 15% or less, stability of a surface film is increased, and the cycle property in the case of using the energy storage device at a high temperature is improved, and hence, such is especially preferred.

In addition, when the nonaqueous solvent includes ethylene carbonate and/or propylene carbonate, resistance of a surface film formed on an electrode becomes small, and hence, such is preferred. A content of ethylene carbonate and/or propylene carbonate is preferably 3% by volume or more, more preferably 5% by volume or more, and still more preferably 7% by volume or more relative to a total volume of the nonaqueous solvent; and an upper limit thereof is preferably 45% by volume or less, more preferably 35% by volume or less, and still more preferably 25% by volume or less.

These solvents may be used solely; in the case where a combination of two or more of the solvents is used, the electrochemical characteristics in the case of using the energy storage device at a high temperature are more improved, and hence, such is preferred; and use of a combination of three or more thereof is especially preferred.

As suitable combinations of these cyclic carbonates, EC and PC; EC and VC; PC and VC; VC and FEC; EC and FEC; PC and FEC; FEC and DFEC; EC and DFEC; PC and DFEC; VC and DFEC; VEC and DFEC; VC and EEC; EC and EEC; EC, PC and VC; EC, PC and FEC; EC, VC and FEC; EC, VC and VEC; EC, VC and EEC; EC, EEC and FEC; PC, VC and FEC; EC, VC and DFEC; PC, VC and DFEC; EC, PC, VC and FEC; EC, PC, VC and DFEC; and the like are preferred. Among the aforementioned combinations, combinations, such as EC and PC; EC and VC; EC and FEC; PC and FEC; EC, PC and VC; EC, PC and FEC; EC, VC and FEC; EC, VC and EEC; EC, EEC and FEC; PC, VC and FEC; EC, PC, VC and FEC; etc., are more preferred.

In addition, a cyclic carbonate containing EC or PC, and a cyclic carbonate having a fluorine atom or an unsaturated bond is preferred; and a cyclic carbonate containing EC or PC, and FEC or VC is still more preferred.

As the linear ester, there are suitably exemplified one or more asymmetric linear carbonates selected from methyl ethyl carbonate (MEC), methyl propyl carbonate (MPC), methyl isopropyl carbonate (MIPC), methyl butyl carbonate, ethyl propyl carbonate, and the like; one or more symmetric linear carbonates selected from dimethyl carbonate (DMC), diethyl carbonate (DEC), dipropyl carbonate, dibutyl carbonate, and the like; and linear carboxylic acid esters, such as pivalic acid esters, such as methyl pivalate (MPV), ethyl pivalate, propyl pivalate, etc., methyl propionate (MP), ethyl propionate (EP), methyl acetate (MA), ethyl acetate (EA), n-propyl acetate (PA), etc. In particular, when the asymmetric linear carbonate is included, the cycle property in the case of using the energy storage device at a high voltage is improved, and the gas generation amount tends to decrease, and hence, such is preferred.

These solvents may be used solely; and in the case of using a combination of two or more of the solvents is used, the cycle property in the case of using the energy storage device at a high temperature is improved, and the gas generation amount decreases, and hence, such is preferred.

Although a content of the linear ester is not particularly limited, it is preferred to use the linear ester in an amount in the range of from 60 to 90% by volume relative to a total volume of the nonaqueous solvent. When the content is 60% by volume or more, and preferably 65% by volume or more, an effect for decreasing the viscosity of the nonaqueous electrolytic solution is thoroughly obtained, whereas when it is 90% by volume or less, preferably 85% by volume or less, and still more preferably 80% by volume or less, an electroconductivity of the nonaqueous electrolytic solution thoroughly increases, whereby the electrochemical characteristics in the case of using the energy storage device at a high temperature are improved, and therefore, it is preferred that the content of the linear ester falls within the aforementioned range.

In addition, in the case of using a linear carbonate, it is preferred to use two or more kinds thereof. Furthermore, it is more preferred that both a symmetric linear carbonate and an asymmetric linear carbonate are included; it is still more preferred that the symmetric linear carbonate includes diethyl carbonate (DEC); it is still more preferred that the asymmetric linear carbonate includes methyl ethyl carbonate (MEC); and it is especially preferred that the linear carbonate includes both diethyl carbonate (DEC) and methyl ethyl carbonate (MEC).

It is preferred that a content of the symmetric linear carbonate is more than a content of the asymmetric linear carbonate.

A proportion of the volume occupied by the symmetric linear carbonate in the linear carbonate is preferably 51% by volume or more, more preferably 55% by volume or more, still more preferably 60% by volume or more, and yet still more preferably 65% by volume or more. An upper limit thereof is preferably 95% by volume or less, more preferably 90% by volume or less, still more preferably 85% by volume or less, and yet still more preferably 80% by volume or less.

The aforementioned case is preferred because the cycle property in the case of using the energy storage device at a high temperature is much more improved.

As for a proportion of the cyclic carbonate and the linear carbonate, from the viewpoint of improving the electrochemical characteristics in the case of using the energy storage device at a high temperature, a ratio of the cyclic carbonate to the linear carbonate (volume ratio) is preferably from 10/90 to 45/55, more preferably from 15/85 to 40/60, and especially preferably from 20/80 to 35/65.

For the purpose of much more improving the electrochemical characteristics in the case of using the energy storage device at a high temperature, it is preferred to further add other additives in the nonaqueous electrolytic solution.

Specifically, examples of other additives include phosphoric acid esters, nitriles, triple bond-containing compounds, S═O bond-containing compounds, acid anhydrides, cyclic phosphazene compounds, diisocyanate compounds, cyclic acetals, aromatic compounds having a branched alkyl group, aromatic compounds, and the like.

Examples of the phosphoric acid ester include trimethyl phosphate, triethyl phosphate, tributyl phosphate, trioctyl phosphate, and the like.

Examples of the nitrile include acetonitrile, propionitrile, succinonitrile, 2-ethylsuccinonitrile, glutaronitrile, 2-methylglutaronitrile, 3-methylglutaronitrile, adiponitrile, pimelonitrile, and the like.

Examples of the triple bond-containing compound include methyl 2-propynyl carbonate, 2-propynyl acetate, 2-propynyl formate, 2-propynyl methacrylate, 2-propynyl methanesulfonate, 2-propynyl vinylsulfonate, di(2-proynyl) oxalate, di(2-propynyl) glutarate, 2-butyne-1,4-diyl dimethanesulfonate, 2-butyne-1,4-diyl diformate, 2-propynyl 2-(diethoxyphosphoryl)acetate, 2-propynyl 2-((methanesulfonyl)oxy)propanoate, and the like.

Examples of the S═O bond-containing compound include sultone compounds, cyclic sulfite compounds, sulfonic acid ester compounds, and the like.

Examples of the sultone compound include 1,3-propanesultone, 1,3-butanesultone, 1,4-butanesultone, 2,4-butanesultone, 1,3-propenesultone, 2,2-dioxide-1,2-oxathiolane-4-yl acetate, 5,5-dimethyl-1,2-oxathiolane-4-one 2,2-dioxide, methylene methanedisulfonate, and the like.

Examples of the cyclic sulfite compound include ethylene sulfite, hexahydrobenzo[1,3,2]dioxathiolane-2-oxide (also called 1,2-cyclohexanediol cyclic sulfite), 5-vinyl-hexahydro-1,3,2-benzodioxathiol-2-oxide, and the like.

Examples of the sulfonic acid ester compound include butane-2,3-diyl dimethanesulfonate, butane-1,4-diyl dimethanesulfonate, methylene methanedisulfonate, dimethyl methanedisulfonate, and the like.

Examples of the vinylsulfone compound include divinylsulfone, 1,2-bis(vinylsulfonyl)ethane, bis(2-vinylsulfonylethyl) ether, vinylsulfonyl fluoride, and the like.

Examples of the acid anhydride include linear carboxylic acid anhydrides, such as acetic anhydride, propionic anhydride, etc., succinic anhydride, maleic anhydride, glutaric anhydride, itaconic anhydride, 3-sulfo-propionic anhydride, and the like.

Examples of the cyclic phosphazene compound include methoxypentafluorocyclotriphosphazene, ethoxypentafluorocyclotriphosphazene, phenoxypentafluorocyclotriphosphazene, ethoxyheptafluorocyclotetraphosphazene, and the like.

Examples of the diisocyanate compound include 1,4-diisocyanatobutane, 1,5-diisocyanatopentane, 1,6-diisocyanatohexane, 1,7-diisocyanatoheptane, and the like.

Examples of the cyclic acetal include 1,3-dioxolane, 1,3-dioxane, and the like.

Examples of the aromatic compound having a branched alkyl group include cyclohexylbenzene, fluorocyclohexylbenzene compounds (e.g., 1-fluoro-2-cyclohexylbenzene, 1-fluoro-3-cyclohexylbenzene, or 1-fluoro-4-cyclohexylbenzene), tert-butylbenzene, tert-amylbenzene, 1-fluoro-4-tert-butylbenzene, and the like.

Examples of the aromatic compound include biphenyl, terphenyl (o-, m-, p-form), diphenyl ether, fluorobenzene, difluorobenzene (o-, m-, p-form), anisole, 2,4-difluoroanisole, partial hydrides of terphenyl (e.g., 1,2-dicyclohexylbenzene, 2-phenylbicyclohexyl, 1,2-diphenylcyclohexane, or o-cyclohexylbiphenyl), and the like.

Above all, when one or more selected from the nitrile, the diisocyanate compound, the cyclic acetal, and the aromatic compound are included, the electrochemical characteristics in the case of using the energy storage device at a high temperature are much more improved, and hence, such is preferred.

Of the nitriles, one or more selected from succinonitrile, 2-ethylsuccinonitrile, glutaronitrile, 2-methylglutaronitrile, 3-methylglutaronitrile, adiponitrile, and pimelonitrile are more preferred.

Of the diisocyanate compounds, one or more selected from 1,5-diisocyanatopentane, 1,6-diisocyanatohexane, and 1,7-diisocyanatoheptane are more preferred.

Of the cyclic acetal compounds, 1,3-dioxane is preferred.

In addition, of the aromatic compounds, one or more selected from biphenyl, terphenyl (o-, m-, p-form), fluorobenzene, cyclohexylbenzene, tert-butylbenzene, and tert-amylbenzene are more preferred.

A content of one or more selected from the nitrile, the diisocyanate compound, the cyclic acetal, and the aromatic compound is preferably from 0.001 to 5% by mass in the nonaqueous electrolytic solution. When the content falls within this range, a surface film is thoroughly formed without becoming excessively thick, and an effect for improving the electrochemical characteristics in the case of using the energy storage device at a high temperature is increased. The content is more preferably 0.005% by mass or more, still more preferably 0.01% by mass or more, and especially preferably 0.03% by mass or more in the nonaqueous electrolytic solution; and an upper limit thereof is more preferably 3% by mass or less, still more preferably 2% by mass or less, and especially preferably 1.5% by mass or less.

In addition, above all, when one or more selected from the triple bond-containing compound, the sultone compound, and the vinylsulfone compound are included, the electrochemical characteristics in the case of using the battery at a high temperature are much more improved, and hence, such is preferred.

Of the triple bond-containing compounds, one or more selected from 2-propynyl methanesulfonate, 2-propynyl vinylsulfonate, di(2-proynyl) oxalate, 2-butyne-1,4-diyl dimethanesulfonate, 2-propynyl 2-(diethoxyphosphoryl)acetate, and 2-propynyl 2-((methanesulfonyl)oxy)propanoate are more preferred.

Of the sultone compounds, one or more selected from 1,3-propanesultone, 1,3-propenesultone, 2,2-dioxide-1,2-oxathiolane-4-yl acetate, 5,5-dimethyl-1,2-oxathiolane-4-one 2,2-dioxide, and methylene methanedisulfonate are more preferred.

Of the vinylsulfone compounds, one or more selected from divinylsulfone, bis(2-vinylsulfonylethyl) ether, and vinylsulfonyl fluoride are more preferred.

A content of one or more selected from the triple bond-containing compound, the sultone compound, and the vinylsulfone compound is preferably from 0.001 to 5% by mass in the nonaqueous electrolytic solution. When the content falls within this range, a surface film is thoroughly formed without becoming excessively thick, and an effect for improving the electrochemical characteristics in the case of using the energy storage device at a high temperature is increased. The content is more preferably 0.005% by mass or more, still more preferably 0.01% by mass or more, and especially preferably 0.03% by mass or more in the nonaqueous electrolytic solution; and an upper limit thereof is more preferably 3% by mass or less, still more preferably 2% by mass or less, and especially preferably 1.5% by mass or less.

In addition, for the purpose of much more improving the electrochemical characteristics in the case of using the energy storage device at a high voltage, it is preferred that the nonaqueous electrolytic solution further includes one or more selected from lithium salts having an oxalic acid skeleton, lithium salts having a phosphoric acid skeleton, and lithium salts having a sulfonic acid skeleton.

As specific examples of the lithium salt, there are suitably exemplified one or more lithium salts having an oxalic acid skeleton, which are selected from lithium bis(oxalate)borate (LiBOB), lithium difluoro(oxalate)borate (LiDFOB), lithium tetrafluoro(oxalate)phosphate (LiTFOP), and lithium difluorobis(oxalate)phosphate (LiDFOP); lithium salts having a phosphoric acid skeleton, such as LiPO₂F₂, Li₂PO₃F, etc.; and one or more lithium salts having a sulfonic acid skeleton, which are selected from lithium trifluoro((methanesulfonyl)oxy)borate (LiTFMSB), lithium pentafluoro((methanesulfonypoxy)phosphate (LiPFMSP), and FSO₃Li. One or more selected from LiBOB, LiDFOB, LiTFOP, LiDFOP, LiPO₂F₂, LiTFMSB, LiPFMSP, and FSO₃Li are more preferred, and LiTFMSB is still more preferred.

A total content of the aforementioned lithium salts, such as LiTFMSB, FSO₃Li, etc., is preferably from 0.001 to 10% by mass in the nonaqueous electrolytic solution. When the content is 10% by mass or less, there is less concern that a surface film is excessively formed on the electrode, thereby causing worsening of the cycle property, and when it is 0.001% by mass or more, a surface film is sufficiently formed, thereby increasing an effect for improving the characteristics in the case of using the battery at a high voltage. The content is preferably 0.05% by mass or more, more preferably 0.1% by mass or more, and still more preferably 0.3% by mass or more in the nonaqueous electrolytic solution; and an upper limit thereof is preferably 5% by mass or less, more preferably 3% by mass or less, and still more preferably 2% by mass or less.

### [Electrolyte Salt]

As the electrolyte salt which is used in the present invention, there are suitably exemplified the following lithium salts.

### (Lithium Salt)

As the lithium salt, there are suitably exemplified one or more lithium salts selected from inorganic lithium salts, such as LiPF₆, LiBF₄, LiN(SO₂F)₂, LiClO₄, etc.; linear fluoroalkyl group-containing lithium salts, such as LiN(SO₂CF₃)₂, LiN(SO₂C₂F₅)₂, LiCF₃SO₃, LiC(SO₂CF₃)₃, LiPF₄(CF₃)₂, LiPF₃(C₂F₅)₃, LiPF₃(CF₃)₃, LiPF₃(iso-C₃F₇)₃, LiPF₅(iso-C₃F₇), etc.; and cyclic fluoroalkylene chain-containing lithium salts, such as (CF₂)₂(SO₂)₂NLi, (CF₂)₃(SO₂)₂NLi, etc.

Of these, one or more selected from LiPF₆, LiBF₄, LiN(SO₂CF₃)₂, LiN(SO₂C₂F₅)₂, and LiN(SO₂F)₂ are preferred; and it is especially preferred to use LiPF₆.

In general, a concentration of the lithium salt that is the electrolyte salt is preferably 0.3 M or more, more preferably 0.7 M or more, and still more preferably 1.1 M or more relative to the aforementioned nonaqueous solvent. In addition, an upper limit thereof is preferably 2.5 M or less, more preferably 2.0 M or less, and still more preferably 1.6 M or less.

In addition, as a suitable combination of these lithium salts, the case where the nonaqueous electrolytic solution includes LiPF₆ and further includes one or more lithium salts selected from LiBF₄, LiN(SO₂CF₃)₂, and LiN(SO₂F)₂ is preferred. When a proportion of the lithium salt other than LiPF₆ in the nonaqueous solvent is 0.001 M or more, an effect for improving the electrochemical characteristics in the case of using the battery at a high temperature is easily exhibited, whereas when it is 0.005 M or less, there is less concern that an effect for improving the electrochemical characteristics in the case of using the battery at a high temperature is worsened, and hence, such is preferred. A proportion of other lithium salt than LiPF₆ is preferably 0.01 M or more, especially preferably 0.03 M or more, and most preferably 0.04 M or more; and an upper limit thereof is preferably 0.4 M or less, and especially preferably 0.2 M or less.

### [Production of Nonaqueous Electrolytic Solution]

The nonaqueous electrolytic solution of the present invention may be, for example, obtained by mixing the aforementioned nonaqueous solvent and adding the phenyl ester compound represented by the general formula (I), in which the benzene ring is substituted with both a halogen atom and a fluoroalkyl group, to the aforementioned electrolyte salt and the nonaqueous electrolytic solution.

At this time, the nonaqueous solvent to be used and the compounds to be added to the nonaqueous electrolytic solution are preferably purified previously to reduce as much as possible the content of impurities, in such an extent that the productivity is not extremely deteriorated.

The nonaqueous electrolytic solution of the present invention may be used in first and second energy storage devices shown below, in which the nonaqueous electrolyte may be used not only in the form of a liquid but also in the form of a gel. Furthermore, the nonaqueous electrolytic solution of the present invention may also be used for a solid polymer electrolyte. Among these, the nonaqueous electrolytic solution is preferably used in the first energy storage device using a lithium salt as the electrolyte salt (i.e., for a lithium battery) or in the second energy storage device (i.e., for a lithium ion capacitor), more preferably used in a lithium battery, and most suitably used in a lithium secondary battery.

### [First Energy Storage Device (Lithium Battery)]

The lithium battery of the present invention is a generic name for a lithium primary battery and a lithium secondary battery. In addition, in the present specification, the term, lithium secondary battery, is used as a concept that includes a so-called lithium ion secondary battery. The lithium battery of the present invention includes a positive electrode, a negative electrode, and the aforementioned nonaqueous electrolytic solution having an electrolyte salt dissolved in a nonaqueous solvent. Other constitutional members used than the nonaqueous electrolytic solution, such as the positive electrode, the negative electrode, etc., are not particularly limited.

For example, as the positive electrode active material for lithium secondary batteries, usable is a complex metal oxide containing lithium and one or more selected from cobalt, manganese, and nickel. These positive electrode active materials may be used solely or in combination of two or more kinds thereof.

As the lithium complex metal oxides, for example, one or more selected from LiCoO₂, LiMn₂O₄, LiNiO₂, LiCo₁₋ₓNiₓO₂ (0.01<x<1), LiCo_{1/3}Ni_{1/3}Mn_{1/3}O₂, LiNi_{1/2}Mn_{3/2}O₄, LiCo_{0.98}Mg_{0.02}O₂, and the like are preferably exemplified. In addition, these materials may be used as a combination, such as a combination of LiCoO₂ and LiMn₂O₄, a combination of LiCoO₂ and LiNiO₂, and a combination of LiMn₂O₄ and LiNiO₂.

In addition, for improving the safety on overcharging and the cycle property, and for enabling the use at a charge potential of 4.3 V or more, a part of the lithium complex metal oxide may be substituted with other elements. For example, a part of cobalt, manganese, or nickel may be substituted with at least one or more elements selected from Sn, Mg, Fe, Ti, Al, Zr, Cr, V, Ga, Zn, Cu, Bi, Mo, La, and the like; or a part of 0 may be substituted with S or F; or the oxide may be coated with a compound containing any of such other elements.

Of those, preferred are lithium complex metal oxides, such as LiCoO₂, LiMn₂O₄, and LiNiO₂, with which the charge potential of the positive electrode in a fully-charged state may be used at 4.3 V or more based on Li; and more preferred are lithium complex metal oxides, such as LiCo₁₋ₓMₓO₂ (wherein M is at least one element selected from Sn, Mg, Fe, Ti, Al, Zr, Cr, V, Ga, Zn, and Cu; and 0.001×0.05), LiCo_{1/3}Ni_{1/3}Mn_{1/3}O₂, LiNi_{1/2}Mn_{3/2}O₄, and a solid solution of Li₂MnO₃ and LiMO₂ (wherein M is a transition metal, such as Co, Ni, Mn, Fe, etc.), that may be used at 4.4 V or more. The use of the lithium complex metal oxide capable of acting at a high charging voltage may easily worsen the electrochemical characteristics particularly in the case of using the battery at a high voltage due to the reaction with the electrolytic solution on charging, but in the lithium secondary battery according to the present invention, the electrochemical characteristics may be prevented from worsening.

Furthermore, a lithium-containing olivine-type phosphate may also be used as the positive electrode active material. Especially preferred are lithium-containing olivine-type phosphates including one or more selected from iron, cobalt, nickel, and manganese. Specific examples thereof include LiFePO₄, LiCoPO₄, LiNiPO₄, LiMnPO₄, and the like.

These lithium-containing olivine-type phosphates may be partly substituted with any other element; and for example, a part of iron, cobalt, nickel, or manganese therein may be substituted with one or more elements selected from Co, Mn, Ni, Mg, Al, B, Ti, V, Nb, Cu, Zn, Mo, Ca, Sr, W, Zr, and the like; or the phosphates may be coated with a compound containing any of these other elements or with a carbon material. Among these, in the case of using a lithium-containing olivine-type phosphate containing at least Co, Ni, or Mn, such as LiCoPO₄, LiNiPO₄, LiMnPO₄, etc., the battery voltage becomes a higher potential, and the effects of the invention of the present application are easily achieved, and hence, such is preferred.

In addition, the lithium-containing olivine-type phosphate may be used, for example, in admixture with the aforementioned positive electrode active material.

In addition, for the positive electrode for lithium primary batteries, there are suitably exemplified oxides or chalcogen compounds of one or more metal elements, such as CuO, Cu₂O, Ag₂O, Ag₂CrO₄, CuS, CuSO₄, TiO₂, TiS₂, SiO₂, SnO, V₂O₅, V₆O₁₂, VOₓ, Nb₂O₅, Bi₂O₃, Bi₂Pb₂O₅, Sb₂O₃, CrO₃, Cr₂O₃, MoO₃, WO₃, SeO₂, MnO₂, Mn₂O₃, Fe₂O₃, FeO, Fe₃O₄, Ni₂O₃, NiO, CoO₃, CoO, etc.; sulfur compounds, such as SO₂, SOCl₂, etc.; and carbon fluorides (graphite fluoride) represented by a general formula (CFₓ)ₙ. Above all, MnO₂, V₂O₅, graphite fluoride, and the like are preferred.

An electroconductive agent for the positive electrode is not particularly limited so long as it is an electron-conductive material that does not undergo a chemical change. Examples thereof include graphites, such as natural graphite (e.g., flaky graphite, etc.), artificial graphite, etc.; carbon blacks, such as acetylene black, Ketjen black, channel black, furnace black, lamp black, thermal black, etc.; and the like. In addition, graphite and carbon black may be properly mixed and used. An addition amount of the electroconductive agent to the positive electrode mixture is preferably from 1 to 10% by mass, and especially preferably from 2 to 5% by mass.

The positive electrode may be produced by mixing the aforementioned positive electrode active material with an electroconductive agent, such as acetylene black, carbon black, etc., and a binder, such as polytetrafluoroethylene (PTFE), polyvinylidene fluoride (PVDF), a copolymer of styrene and butadiene (SBR), a copolymer of acrylonitrile and butadiene (NBR), carboxymethyl cellulose (CMC), an ethylene-propylene-diene terpolymer, etc., adding a high-boiling point solvent, such as 1-methyl-2-pyrrolidone, etc., thereto, followed by kneading to prepare a positive electrode mixture, applying this positive electrode mixture onto a collector, such as an aluminum foil, a stainless steel-made lath plate, etc., and drying and shaping the resultant under pressure, followed by a heat treatment in vacuum at a temperature of from about 50° C. to 250° C. for about 2 hours.

A density of a portion of the positive electrode except for the collector is generally 1.5 g/cm³ or more, and for the purpose of further increasing the capacity of the battery, the density is preferably 2 g/cm³ or more, more preferably 3 g/cm³ or more, and still more preferably 3.6 g/cm³ or more. An upper limit thereof is preferably 4 g/cm³ or less.

As the negative electrode active material for lithium secondary batteries, one or more selected from a lithium metal, lithium alloys, carbon materials capable of absorbing and releasing lithium [e.g., graphitizable carbon, non-graphitizable carbon having a spacing of the (002) plane of 0.37 nm or more, graphite having a spacing of the (002) plane of 0.34 nm or less, etc.], tin (elemental substance), tin compounds, silicon (elemental substance), silicon compounds, and lithium titanate compounds, such as Li₄Ti₅O₁₂, etc., may be used in combination.

Of those, in absorbing and releasing ability of a lithium ion, it is more preferred to use a high-crystalline carbon material, such as artificial graphite, natural graphite, etc.; and it is especially preferred to use a carbon material having a graphite-type crystal structure in which a lattice (002) spacing (d₀₀₂) is 0.340 nm (nanometers) or less, and especially from 0.335 to 0.337 nm.

By using an artificial graphite particle having a bulky structure in which plural flat graphite fine particles are mutually gathered or bound in non-parallel, or a graphite particle prepared by, for example, subjecting a flaky natural graphite particle to a spheroidizing treatment by repeatedly giving a mechanical action, such as compression force, frictional force, shear force, etc., when a ratio [I(110)/I(004)] of a peak intensity I(110) of the (110) plane to a peak intensity I(004) of the (004) plane of the graphite crystal, which is obtained from the X-ray diffraction measurement of a negative electrode sheet at the time of shaping under pressure of a portion of the negative electrode except for the collector in a density of 1.5 g/cm³ or more, is 0.01 or more, the electrochemical characteristics in a much broader temperature range are improved, and hence, such is preferable; and the peak intensity ratio [I(110)/I(004)] is more preferably 0.05 or more, and still more preferably 0.1 or more. In addition, when excessively treated, there may be the case where the crystallinity is worsened, and the discharge capacity of the battery is worsened, and therefore, an upper limit thereof is preferably 0.5 or less, and more preferably 0.3 or less.

In addition, when the high-crystalline carbon material (core material) is coated with a carbon material that is more low-crystalline than the core material, the electrochemical characteristics in the case of using the battery at a high voltage become much more favorable, and hence, such is preferable. The crystallinity of the carbon material of the coating may be confirmed by TEM.

When the high-crystalline carbon material is used, there is a tendency that it reacts with the nonaqueous electrolytic solution on charging, thereby worsening the electrochemical characteristics at low temperatures or high temperatures due to an increase of the interfacial resistance; however, in the lithium secondary battery according to the present invention, the electrochemical characteristics in the case of using the battery at a high temperature become favorable.

In addition, as the metal compound capable of absorbing and releasing lithium, serving as a negative electrode active material, there are preferably exemplified compounds containing at least one metal element selected from Si, Ge, Sn, Pb, P, Sb, Bi, Al, Ga, In, Ti, Mn, Fe, Co, Ni, Cu, Zn, Ag, Mg, Sr, Ba, etc. The metal compound may be in any form including an elemental substance, an alloy, an oxide, a nitride, a sulfide, a boride, an alloy with lithium, and the like, and any of an elemental substance, an alloy, an oxide, and an alloy with lithium is preferred because the battery capacity may be increased thereby. Above all, more preferred are those containing at least one element selected from Si, Ge, and Sn, and especially preferred are those containing one or more elements selected from Si and Sn, as capable of increasing the battery capacity.

In the case of mixing the metal compound capable of absorbing and releasing lithium with the carbon material and using the mixture as the negative electrode active material for the negative electrode, as for a ratio of the metal compound capable of absorbing and releasing lithium and the carbon material, from the viewpoint of a cycle improvement on the basis of an effect for improving an electron conductivity due to the mixing with the carbon material, an amount of the carbon material is preferably 10% by mass or more, and more preferably 30% by mass or more relative to a total mass of the metal compound capable of absorbing and releasing lithium in the negative electrode mixture. In addition, when the ratio of the carbon material with which the metal compound capable of absorbing and releasing lithium is mixed is too large, there is a concern that the amount of the metal compound capable of absorbing and releasing lithium in the negative electrode mixture is decreased, whereby an effect for increasing the battery capacity becomes small, and therefore, the amount of the carbon material is preferably 98% by mass or less, and more preferably 90% by mass or less relative to a total mass of the metal compound capable of absorbing and releasing lithium.

In the case of using a combination of the nonaqueous electrolytic solution containing the phenyl ester compound represented by the general formula (I), in which the benzene group is substituted with both a halogen atom and a fluoroalkyl group, according to the present invention and the aforementioned negative electrode using a mixture of the aforementioned metal compound capable of absorbing and releasing lithium and the carbon material as the negative electrode active material, it may be considered that in view of the fact that the phenyl ester compound represented by the general formula (I) acts on both the metal compound and the carbon material, the electrical contact of the metal compound in which a volume change following absorption and release of lithium is generally large, with the carbon material is reinforced, whereby the cycle property is much more improved.

The negative electrode may be formed in such a manner that the same electroconductive agent, binder, and high-boiling point solvent as in the formation of the aforementioned positive electrode are used and kneaded to provide a negative electrode mixture, and the negative electrode mixture is then applied onto a collector, such as a copper foil, etc., dried, shaped under pressure, and then heat-treated in vacuum at a temperature of from about 50° C. to 250° C. for about 2 hours.

A density of the portion of the negative electrode except for the collector is generally 1.1 g/cm³ or more, and for further increasing the battery capacity, the density is preferably 1.5 g/cm³ or more, and especially preferably 1.7 g/cm³ or more. An upper limit thereof is preferably 2 g/cm³ or less.

In addition, examples of the negative electrode active material for lithium primary batteries include a lithium metal and a lithium alloy.

The structure of the lithium battery is not particularly limited, and may be a coin-type battery, a cylinder-type battery, a prismatic battery, a laminate-type battery, or the like, each having a single-layered or multi-layered separator.

Although the separator for the battery is not particularly limited, a single-layered or laminated micro-porous film of a polyolefin, such as polypropylene, polyethylene, etc., as well as a woven fabric, a nonwoven fabric, or the like may be used.

The lithium secondary battery in the present invention has excellent electrochemical characteristics even in the case where the final charging voltage of the positive electrode against the lithium metal is 4.2 V or more, and particularly 4.3 V or more, and furthermore, the characteristics thereof are still favorable even at 4.4 V or more. Although a current value is not particularly limited, in general, the battery is used within the range of from 0.1 to 30 C. In addition, the lithium battery in the present invention may be charged and discharged at from −40 to 100° C., and preferably from −10 to 80° C.

In the present invention, as a countermeasure against an increase in the internal pressure of the lithium battery, such a method may be employed that a safety valve is provided in the battery cap, and a cutout is provided in the battery component, such as a battery can, a gasket, etc. In addition, as a safety countermeasure for preventing overcharging, a current cut-off mechanism capable of detecting an internal pressure of the battery to cut off the current may be provided in a battery cap.

### [Second Energy Storage Device (Lithium Ion Capacitor)]

The second energy storage device is an energy storage device that stores energy by utilizing intercalation of a lithium ion into a carbon material, such as graphite, etc., which is the negative electrode. This energy storage device is called a lithium ion capacitor (LIC). Examples of the positive electrode include one utilizing an electric double layer between an active carbon electrode and an electrolytic solution, one utilizing a doping/dedoping reaction of a n-conjugated polymer electrode, and the like. The electrolytic solution contains at least a lithium salt, such as LiPF₆, etc.

The nonaqueous electrolytic solution of the present invention is capable of improving charging and discharging properties of a lithium ion capacitor which is used at a high voltage.

The phenyl ester compound of the present invention, in which the benzene ring is substituted with both a halogen atom and a fluoroalkyl group, that is a novel compound, is represented by the following general formula (II).

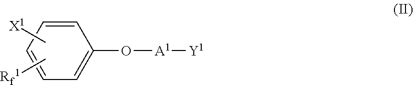

(In the formula, Re represents a fluoroalkyl group having 1 to 6 carbon atoms; and X¹ represents a halogen atom. A¹ has a structure represented by —S(═O)₂—, —C(═O)—, —C(═O)—O—, —C(═O)-L³-C(═O)—, —C(═O)-L⁴-P(═O)(OR¹)—O—, or —P(═O)(OR¹)—O—. Y¹ represents a fluorine atom, a hydrogen atom, an alkyl group having 1 to 6 carbon atoms, an alkenyl group having 2 to 6 carbon atoms, an alkynyl group having 3 to 6 carbon atoms, or an aryl group having 6 to 12 carbon atoms; L³ represents an alkylene group having 1 to 8 carbon atoms, an alkenylene group having 2 to 8 carbon atoms, an alkynylene group having 2 to 8 carbon atoms, or a direct bond; L⁴ represents an alkylene group having 1 to 8 carbon atoms; and R¹ represents an alkyl group having 1 to 6 carbon atoms. However, only when A¹ is —S(═O)₂—, Y¹ may be a fluorine atom; and only when A¹ is —C(═O)—, Y¹ may be a hydrogen atom. However, the case where A¹ is —S(═O)₂— and Y¹ is a trifluoromethyl group is excluded.

At least one hydrogen atom in each group of the aforementioned alkyl group, alkenyl group, alkynyl group, aryl group, alkylene group, alkenylene group, and alkynylene group may be substituted with a halogen atom.)

In the general formula (II), the halogen atom as the substituent X¹ is preferably a fluorine atom or a bromine atom, and more preferably a fluorine atom.

With respect to the substituent Re, the substituent A¹, the substituent Y¹, the substituent L³, the substituent L⁴, and the substituent R¹, explanations thereof are the same as those in the foregoing general formula (I), and suitable examples thereof are also the same. Thus, in order to avoid duplication, the explanations are omitted. In this case, the substituents R_{f}, A, Y, L¹, L², and R in the general formula (I) correspond to the substituents R_{f}¹, A¹, Y¹, L³, L⁴, and 1 in the general formula (II), respectively.

The phenyl ester compound of the present invention may be synthesized by the following methods (a) to (c), but it should not be construed that the present invention is limited to these methods.

(a) A method of allowing a phenol compound in which the benzene ring is substituted with both a halogen atom and a fluoroalkyl group (hereinafter referred to simply as “phenol compound”) to react with at least one compound corresponding to the -A¹-Y¹ group in the general formula (II), which is selected from an alkylsulfonyl halide, an alkenylsulfo halide, an alkynylsulfo halide, arylsulfo halide, an alkylcarbonyl halide, an alkenylcarbonyl halide, an alkynylcarbonyl halide, an arylcarbonyl halide, an alkoxycarbonyl halide, an alkenyloxycarbonyl halide, an alkynyloxycarbonyl halide, an aryloxycarbonyl halide, an oxalyl dihalide, and a dialkoxyphosphorylalkylcarbonyl halide (hereinafter referred to simply as “halide compound”) in the presence or absence of a solvent and in the presence or absence of a base (hereinafter also referred to as “method (a)”).
(b) A method of allowing the aforementioned phenol compound to react with a carbonylating agent in the presence or absence of a solvent (hereinafter also referred to as “method (b)”).
(c) A method of condensing the aforementioned phenol compound with a carboxylic acid compound corresponding to the -A¹-Y¹ group in the general formula (II) in the presence or absence of a solvent and in the presence of an acid catalyst or a dehydrating agent (hereinafter also referred to as “method (c)”).
[Method (a)]

The method (a) is a method of allowing the aforementioned phenol compound to react with the aforementioned halide compound in the presence or absence of a solvent and in the presence or absence of a base. It is to be noted that as for the phenol compound and the halide compound, commercially available products may be used, or these compounds may also be synthesized by existent methods.

In the method (a), a use amount of the halide compound is preferably 0.8 to 20 moles, more preferably 0.9 to 10 moles, and still more preferably 1 to 5 moles per mole of the phenol compound.

Examples of the halide compound which is used for the method (a) include methanesulfonyl chloride, 4-methylbenzenesulfonyl chloride, methyl chloroformate, ethyl chloroformate, vinyl chloroformate, 2-propenyl chloroformate, 2-propynyl chloroformate, phenyl chloroformate, 4-methylphenyl chloroformate, 4-fluorophenyl chloroformate, 2-(dimethoxyphosphoryl)acetyl chloride, 2-(diethoxyphosphoryDacetyl chloride, and the like.

In the reaction of the method (a), though the reaction proceeds in the absence of a solvent, the solvent may be used so long as it is inert to the reaction. Examples of the solvent which is used include aliphatic hydrocarbons, such as heptane, cyclohexane, etc.; halogenated hydrocarbons, such as dichloromethane, dichloroethane, etc.; aromatic hydrocarbons, such as toluene, xylene, etc., halogenated aromatic hydrocarbons, such as chlorobenzene, fluorobenzene, etc.; ethers, such as diisopropyl ether, dioxane, dimethoxyethane, etc.; esters, such as ethyl acetate, butyl acetate, dimethyl carbonate, diethyl carbonate, etc.; nitriles, such as acetonitrile, propionitrile, etc.; sulfoxides, such as dimethyl sulfoxide, sulfolane, etc.; amides, such as N,N-dimethylformamide, N,N-dimethylacetamide, etc.; and mixtures thereof. Of these, aliphatic or aromatic hydrocarbons and esters, such as heptane, cyclohexane, toluene, ethyl acetate, dimethyl carbonate, etc., are preferred.

A use amount of the solvent is preferably 0 to 30 parts by mass, and more preferably 1 to 10 parts by mass per part by mass of the phenol compound.

In the reaction of the method (a), though the reaction proceeds in the absence of a base, if the base is allowed to coexist, the reaction is promoted, and hence, such is preferred. Any of inorganic bases and organic bases may be used as the base.

Examples of the inorganic base include potassium carbonate, sodium carbonate, calcium hydroxide, calcium oxide, and the like. Examples of the organic base include linear or branched aliphatic tertiary amines and unsubstituted or substituted imidazoles, pyridines, and pyrimidines. Of these, trialkylamines, such as trimethylamine, triethylamine, tripropylamine, tributylamine, diisopropylethylamine, etc.; and pyridines, such as pyridine, N,N-dimethylaminopyridine, etc. are preferred.

A use amount of the base is preferably 0.8 to 5 moles, more preferably 1 to 3 moles, and still more preferably 1 to 1.5 moles per mole of the phenol compound.

In the reaction of the method (a), from the viewpoint of not lowering the reactivity, a lower limit of a reaction temperature is preferably −20° C. or higher, and more preferably −10° C. or higher. In addition, from the viewpoint of inhibiting a side reaction or decomposition of the product, an upper limit of the reaction temperature is preferably 80° C. or lower, and more preferably 50° C. or lower.

While a reaction time may be properly changed depending upon the reaction temperature or a scale, if the reaction time is too short, unreacted materials remain, whereas conversely, if the reaction time is too long, there is a concern that decomposition of the reaction product or a side reaction is generated. Thus, the reaction time is preferably 0.1 to 12 hours, and more preferably 0.2 to 6 hours.

[Method (b)]

The method (b) is a method of allowing the aforementioned phenol compound to react with a carbonylating agent in the presence or absence of a solvent.

In the reaction of the method (b), a use amount of the carbonylating agent is preferably 0.4 to 5 moles, more preferably 0.5 to 3 moles, and still more preferably 0.5 to 1 mole per mole of the phenol compound.

Examples of the carbonylating agent which is used for the method (b) include N,N′-carbonyl diimidazole, phenyl chloroformate, triphosgene, and the like.

In the reaction of the method (b), though the reaction proceeds in the absence of a solvent, the solvent may be used so long as it is inert to the reaction. Examples of the solvent which is used include the same solvents described in the method (a), inclusive of aliphatic hydrocarbons, halogenated hydrocarbons, aromatic hydrocarbons, halogenated aromatic hydrocarbons, ethers, esters, nitriles, sulfoxides, amides, and mixtures thereof. Of these, aliphatic or aromatic hydrocarbons which are hardly miscible with water, such as heptane, cyclohexane, toluene, etc., are preferred.

A use amount of the solvent is preferably 0 to 30 parts by mass, and more preferably 1 to 10 parts by mass per part by mass of the phenol compound.

In the reaction of the method (b), though the reaction proceeds in the absence of a base, if the base is allowed to coexist, the reaction is promoted, and hence, such is preferred. Any of inorganic bases and organic bases may be used as the base.

As the inorganic base and the organic base, the same bases as explained in the method (a) are preferably exemplified.

A use amount of the base is preferably 0.8 to 5 moles, more preferably 1 to 3 moles, and still more preferably 1 to 1.5 moles per mole of the phenol compound.

In the reaction of the method (b), a lower limit of a reaction temperature is preferably −20° C. or higher, and from the viewpoint of not lowering the reactivity, it is more preferably 0° C. or higher. In addition, from the viewpoint of inhibiting a side reaction or decomposition of the product, an upper limit of the reaction temperature is preferably 80° C. or lower, and more preferably 50° C. or lower.

While a reaction time of the method (b) may be properly changed depending upon the reaction temperature or a scale, if the reaction time is too short, unreacted materials remain, whereas conversely, if the reaction time is too long, there is a concern that decomposition of the reaction product or a side reaction is generated. Thus, the reaction time is preferably 0.1 to 24 hours, and more preferably 0.2 to 12 hours.

[Method (c)]

The method (c) is a method of condensing the aforementioned phenol compound with a carboxylic acid compound corresponding to the -A¹-Y¹ group in the general formula (II) in the presence or absence of a solvent and in the presence or absence of an acid catalyst or a dehydrating agent.

In the reaction of the method (c), a use amount of the carboxylic acid compound is preferably 0.8 to 20 moles, more preferably 0.9 to 10 moles, and still more preferably 1 to 5 moles per mole the phenol compound.

Examples of the carboxylic acid compound which is used for the method (c) include formic acid, acetic acid, 2-(diethoxyphosphoryl)acetic acid, and the like.

In the reaction of the method (c), though the reaction proceeds in the absence of a solvent, the solvent may be used so long as it is inert to the reaction. Examples of the solvent which is used include the same solvents described in the method (a), inclusive of aliphatic hydrocarbons, halogenated hydrocarbons, aromatic hydrocarbons, halogenated aromatic hydrocarbons, ethers, esters, nitriles, sulfoxides, amides, and mixtures thereof. Of these, aliphatic or aromatic hydrocarbons, such as heptane, cyclohexane, toluene, etc., are preferred.

A use amount of the solvent is preferably 0 to 30 parts by mass, and more preferably 1 to 10 parts by mass per part by mass of the phenol compound.

In the method (c), in the case of using an acid catalyst, examples of the acid catalyst which may be used include mineral acids, such as sulfuric acid, phosphoric acid, etc.; sulfonic acids, such as p-toluenesulfonic acid, methanesulfonic acid, trifluoromethanesulfonic acid, etc.; Lewis acids, such as trifluoroboric acid, tetraisopropoxytitanium, etc.; solid acids, such as zeolite, acidic resins, etc.; and mixtures thereof. Of these, sulfonic acids, such as p-toluenesulfonic acid, methanesulfonic acid, trifluoromethanesulfonic acid, etc.; and Lewis acids, such as tetraisopropoxytitanium, etc., are preferred.

From the viewpoint of inhibiting a side reaction, a use amount of the catalyst is preferably 0.001 to 5 moles, more preferably 0.01 to 1 mole, and still more preferably 0.01 to 0.3 moles per mole of the phenol compound.

In addition, in the case of using a dehydrating agent, as the dehydrating agent which can be used, there are exemplified one or more selected from dicyclo hexyl carbodiimide, 1-ethyl-3-(3-dimethylaminopropyl) carbodiimide hydrochloride (WSC), N,N′-carbonyl diimidazole, di-2-pyridyl carbonate, phenyl dichlorophosphate, a mixture of diethylazodicarboxylic acid ethyl and triphenylphosphine, and the like.

A use amount of the dehydrating agent is preferably 0.8 to 10 moles, more preferably 0.9 to 5 moles, and still more preferably 1 to 3 moles per mole of the phenol compound.

In the reaction of the method (c), in the case of using the acid catalyst, a lower limit of the reaction temperature is preferably 0° C. or higher, and from the viewpoint of not lowering the reactivity, it is more preferably 20° C. or higher. In addition, from the viewpoint of inhibiting a side reaction or decomposition of the product, an upper limit of the reaction temperature is preferably 200° C. or lower, and more preferably 150° C. or lower.

In addition, in the case of using the dehydrating agent, a lower limit of the reaction temperature is −20° C. or higher, and from the viewpoint of not lowering the reactivity, it is more preferably 0° C. or higher. In addition, from the viewpoint of inhibiting a side reaction or decomposition of the product, an upper limit of the reaction temperature is preferably 100° C. or lower, and more preferably 50° C. or lower.

While a reaction time of the method (c) may be properly changed depending upon the reaction temperature or a scale, if the reaction time is too short, unreacted materials remain, whereas conversely, if the reaction time is too long, there is a concern that decomposition of the reaction product or a side reaction is generated. Thus, the reaction time is preferably 0.1 to 24 hours, and more preferably 0.2 to 12 hours.

### EXAMPLES

Synthesis Examples of a cyclic sulfonic acid ester compound which is used in the present invention are hereunder described, but it should not be construed that the present invention is limited to these Synthesis Examples.

### Synthesis Example 1

### Synthesis of 4-Fluoro-3-(trifluoromethyl)phenyl Methanesulfonate (Structural Formula A2)

10.00 g (55.5 mmoles) of 4-fluoro-3-(trifluoromethyl)phenol and 6.87 g (60.0 mmoles) of methanesulfonyl chloride were dissolved in 50 mL of dimethyl carbonate, followed by cooling to 2° C. To this solution, 6.07 g (60.0 mmoles) of triethylamine was added dropwise at 2 to 11° C. over 15 minutes, and the mixture was stirred at room temperature for one hour. After confirming vanishment of the raw materials by gas chromatography, the reaction liquid was washed with water and subjected to liquid separation, and the organic layer was then concentrated. The residue was purified by distillation under reduced pressure, thereby obtaining 6.87 g (yield: 48%) of desired 4-fluoro-3-(trifluoromethyl)phenyl methanesulfonate as a colorless liquid.

The obtained 4-fluoro-3-(trifluoromethyl)phenyl methanesulfonate was subjected to ¹H-NMR measurement, thereby confirming a structure thereof. The results are shown below.

¹H-NMR (300 MHz, CDCl₃): δ=7.58-7.46 (m, 2H), 7.33-7.23 (m, 1H), 3.22 (s, 3H)

### Synthesis Example 2

### Synthesis of 4-Fluoro-3-(trifluoromethyl)phenyl Acetate (Structural Formula B2)

5.00 g (27.8 mmoles) of 4-fluoro-3-(trifluoromethyl)phenol and 3.09 g (30.5 mmoles) of triethylamine were dissolved in 30 mL of dimethyl carbonate, followed by cooling to 5° C. To this solution, 2.39 g (30.5 mmoles) of acetyl chloride was added dropwise at 5 to 16° C. over 10 minutes, and the mixture was stirred at room temperature for one hour. After confirming vanishment of the raw materials by gas chromatography, the reaction liquid was washed with water and subjected to liquid separation, and the organic layer was then concentrated. The resulting concentrated liquid was purified by silica gel column chromatography (WAKOGEL C-200, elution with hexane/ethyl acetate=9/1), thereby obtaining 5.77 g (yield: 93%) of desired 4-fluoro-3-(trifluoromethyl)phenyl acetate as a colorless liquid.

The obtained 4-fluoro-3-(trifluoromethyl)phenyl acetate was subjected to ¹H-NMR measurement, thereby confirming a structure thereof. The results are shown below.

¹H-NMR (300 MHz, CDCl₃): δ=7.38-7.26 (m, 2H), 7.23-7.18 (m, 1H), 2.31 (s, 3H)

### Synthesis Example 3

### Synthesis of 4-Fluoro-3-(trifluoromethyl)phenyl Methyl Carbonate (Structural Formula C1)

5.00 g (27.8 mmoles) of 4-fluoro-3-(trifluoromethyl)phenol and 3.09 g (30.5 mmoles) of triethylamine were dissolved in 30 mL of dimethyl carbonate, followed by cooling to 5° C. To this solution, 2.88 g (30.5 mmoles) of methyl chloroformate was added dropwise at 5 to 14° C. over 10 minutes, and the mixture was stirred at room temperature for one hour. After confirming vanishment of the raw materials by gas chromatography, the reaction liquid was washed with water and subjected to liquid separation, and the organic layer was then concentrated. The resulting concentrated liquid was purified by silica gel column chromatography (WAKOGEL C-200, elution with hexane/ethyl acetate=9/1), thereby obtaining 6.28 g (yield: 95%) of desired 4-fluoro-3-(trifluoromethyl)phenyl methyl carbonate as a colorless liquid.

The obtained 4-fluoro-3-(trifluoromethyl)phenyl methyl carbonate was subjected to ¹H-NMR measurement, thereby confirming a structure thereof. The results are shown below.

¹H-NMR (300 MHz, CDCl₃): δ=7.46-7.35 (m, 2H), 7.26-7.20 (m, 1H), 3.93 (s, 3H)

### Synthesis Example 4

### Synthesis of Bis(4-fluoro-3-(trifluoromethyl)phenyl) Oxalate (Structural Formula D11)

5.00 g (27.8 mmoles) of 4-fluoro-3-(trifluoromethyl)phenol and 3.09 g (30.5 mmoles) of triethylamine were dissolved in 30 mL of dimethyl carbonate, followed by cooling to 5° C. To this solution, 1.76 g (13.9 mmoles) of oxalyl chloride was added dropwise at 5 to 18° C. over 10 minutes, and the mixture was stirred at room temperature for one hour. After confirming vanishment of the raw materials by gas chromatography, the reaction liquid was washed with water and subjected to liquid separation, and the organic layer was then concentrated. The resulting concentrated liquid was purified by silica gel column chromatography (WAKOGEL C-200, elution with hexane/ethyl acetate=9/1), thereby obtaining 0.96 g (yield: 17%) of desired bis(4-fluoro-3-(trifluoromethyl)phenyl) oxalate as a colorless liquid.

The obtained bis(4-fluoro-3-(trifluoromethyl)phenyl) oxalate was subjected to ¹H-NMR measurement, thereby confirming a structure thereof. The results are shown below.

¹H-NMR (300 MHz, CDCl₃): δ=7.40-7.27 (m, 2H), 7.25-7.20 (m, 1H)

### Synthesis Example 5

### Synthesis of 4-Fluoro-3-(trifluoromethyl)phenyl Diethylphosphate (Structural Formula F2)

8.54 g (47.4 mmoles) of 4-fluoro-3-(trifluoromethyl)phenol and 5.28 g (52.1 mmoles) of triethylamine were dissolved in 50 mL of dimethyl carbonate, followed by cooling to 5° C. To this solution, 9.00 g (52.1 mmoles) of diethyl chlorophosphate was added dropwise at 5 to 13° C. over 15 minutes, and the mixture was stirred at room temperature for 3 hours. After confirming vanishment of the raw materials by gas chromatography, the reaction liquid was washed with water and subjected to liquid separation, and the organic layer was then concentrated. The resulting concentrated liquid was purified by silica gel column chromatography (WAKOGEL C-200, elution with hexane/ethyl acetate=4/1), thereby obtaining 3.80 g (yield: 92%) of desired 4-fluoro-3-(trifluoromethyl)phenyl diethylphosphate as a pale yellow liquid.

The obtained 4-fluoro-3-(trifluoromethyl)phenyl diethylphosphate was subjected to ¹H-NMR measurement, thereby confirming a structure thereof. The results are shown below.

¹H-NMR (300 MHz, CDCl₃): δ=7.47-7.42 (m, 211), 7.21-7.15 (m, 111), 4.29-4.19 (m, 2H), 1.44-1.33 (m, 3H)

### Examples 1 to 40 and Comparative Examples 1 to 3

### Production of Lithium Ion Secondary Battery

94% by mass of LiNi_{1/3}Mn_{1/3}Co_{1/3}O₂ and 3% by mass of acetylene black (electroconductive agent) were mixed and then added to and mixed with a solution which had been prepared by dissolving 3% by mass of polyvinylidene fluoride (binder) in 1-methyl-2-pyrrolidone in advance, thereby preparing a positive electrode mixture paste. This positive electrode mixture paste was applied onto one surface of an aluminum foil (collector), dried, and treated under pressure, followed by cutting into a predetermined size, thereby producing a stripe-like positive electrode sheet. A density of a portion of the positive electrode except for the collector was 3.6 g/cm³. In addition, 10% by mass of silicon (elemental substance), 80% by mass of artificial graphite (d₀₀₂=0.335 nm, negative electrode active material), and 5% by mass of acetylene black (electroconductive agent) were mixed and then added to and mixed with a solution which had been prepared by dissolving 5% by mass of polyvinylidene fluoride (binder) in 1-methyl-2-pyrrolidone in advance, thereby preparing a negative electrode mixture paste. This negative electrode mixture paste was applied onto one surface of a copper foil (collector), dried, and treated under pressure, followed by cutting into a predetermined size, thereby producing a negative electrode sheet. A density of a portion of the negative electrode except for the collector was 1.5 g/cm³. In addition, this electrode sheet was used and analyzed by means of X-ray diffraction, and as a result, a ratio [I(110)/I(004)] of a peak intensity I(110) of the (110) plane to a peak intensity I(004) of the (004) plane of the graphite crystal was found to be 0.1.

The above-obtained positive electrode sheet, a micro-porous polyethylene film-made separator, and the above-obtained negative electrode sheet were laminated in this order, and a nonaqueous electrolytic solution having each of compositions shown in Tables 1 and 2 was added thereto, thereby producing a laminate-type battery.

### [Evaluation of High-Voltage Cycle Property]

In a thermostatic chamber at 60° C., the battery produced by the aforementioned method was treated by repeating a cycle of charging up to a final voltage of 4.3 V with a constant current of 1 C and under a constant voltage for 3 hours and subsequently discharging down to a discharging voltage of 3.0 V with a constant current of 1 C, until it reached 100 cycles. Then, a discharge capacity retention rate was determined according to the following equation.

Discharge capacity retention rate (%)=(Discharge capacity after 100 cycles)/(Discharge capacity at 1st cycle)×100

[Evaluation of Gas Generation Amount after 100 Cycles]

A gas generation amount after 100 cycles was measured by the Archimedean method. As for the gas generation amount, a relative gas generation amount was examined on the basis of defining the gas generation amount of Comparative Example 1 as 100%.

In addition, the production condition and battery characteristics of each of the batteries are shown in Tables 1 to 3.

TABLE 1

| | | Phenyl ester compound | | | |
|---|---|---|---|---|---|
| | | | Addition | Discharge | |
| | | | amount | capacity | Gas |
| | Composition of electrolyte | | (content in | retention | generation |
| | salt | | nonaqueous | rate after | amount |
| | Composition of nonaqueous | | electrolytic | cycle at | after cycle |
| | electrolytic solution | | solution) | 60° C. | at 60° C. |
| | (volume ratio of solvent) | Kind | (% by mass) | (%) | (%) |
| Example 1 | 1.2M LiPF₆ EC/MEC/DEC (30/30/40) | 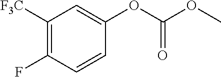 [SMILES: COC(=O)Oc1ccc(F)c(C(F)(F)F)c1] | 1 | 76 | 72 |
| Example 2 | 1.2M LiPF₆ EC/VC/MEC/DEC (29/1/30/40) | 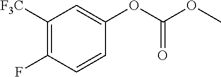 [SMILES: COC(=O)Oc1ccc(F)c(C(F)(F)F)c1] | 0.05 | 75 | 73 |
| Example 3 | 1.2M LiPF₆ EC/VC/MEC/DEC (29/1/30/40) | 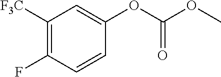 [SMILES: COC(=O)Oc1ccc(F)c(C(F)(F)F)c1] | 1 | 79 | 70 |
| Example 4 | 1.2M LiPF₆ EC/VC/MEC/DEC (29/1/30/40) | 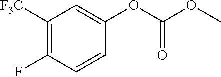 [SMILES: COC(=O)Oc1ccc(F)c(C(F)(F)F)c1] | 3 | 78 | 66 |
| Example 5 | 1.2M LiPF₆ EC/VC/MEC/DEC (29/1/30/40) | 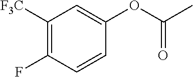 | 1 | 75 | 72 |
| Example 6 | 1.2M LiPF₆ EC/VC/MEC/DEC (29/1/30/40) | 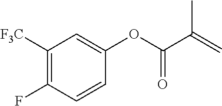 [SMILES: C=C(C)C(=O)Oc1ccc(F)c(C(F)(F)F)c1] | 1 | 77 | 69 |
| Example 7 | 1.2M LiPF₆ EC/VC/MEC/DEC (29/1/30/40) | 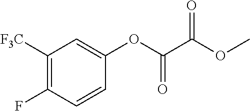 [SMILES: COC(=O)C(=O)Oc1ccc(F)c(C(F)(F)F)c1] | 1 | 78 | 74 |
| Example 8 | 1.2M LiPF₆ EC/VC/MEC/DEC (29/1/30/40) | 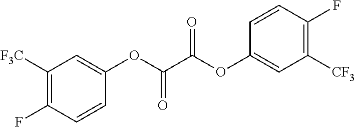 [SMILES: O=C(Oc1ccc(F)c(C(F)(F)F)c1)C(=O)Oc1ccc(F)c(C(F)(F)F)c1] | 1 | 77 | 71 |
| Example 9 | 1.2M LiPF₆ EC/VC/MEC/DEC (29/1/30/40) | 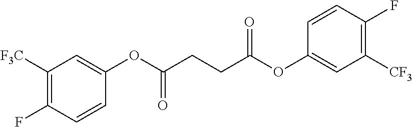 [SMILES: O=C(CCC(=O)Oc1ccc(F)c(C(F)(F)F)c1)Oc1ccc(F)c(C(F)(F)F)c1] | 1 | 81 | 65 |
| Example 10 | 1.2M LiPF₆ EC/VC/MEC/DEC (29/1/30/40) | 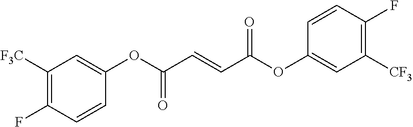 [SMILES: O=C(/C=C/C(=O)Oc1ccc(F)c(C(F)(F)F)c1)Oc1ccc(F)c(C(F)(F)F)c1] | 1 | 83 | 63 |
| Example 11 | 1.2M LiPF₆ EC/VC/MEC/DEC (29/1/30/40) | 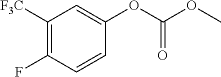 [SMILES: COC(=O)Oc1ccc(F)c(C(F)(F)F)c1] | 1 | 79 | 70 |
| Example 12 | 1.2M LiPF₆ EC/VC/MEC/DEC (29/1/30/40) | 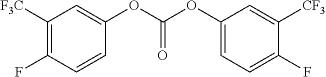 [SMILES: O=C(Oc1ccc(F)c(C(F)(F)F)c1)Oc1ccc(F)c(C(F)(F)F)c1] | 1 | 80 | 64 |
| Example 13 | 1.2M LiPF₆ EC/VC/MEC/DEC (29/1/30/40) | 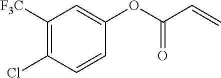 [SMILES: C=CC(=O)Oc1ccc(Cl)c(C(F)(F)F)c1] | 1 | 75 | 71 |
| Example 14 | 1.2M LiPF₆ EC/VC/MEC/DEC (29/1/30/40) | 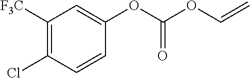 [SMILES: C=COC(=O)Oc1ccc(Cl)c(C(F)(F)F)c1] | 1 | 78 | 67 |
| Example 15 | 1.2M LiPF₆ EC/VC/MEC/DEC (29/1/30/40) | 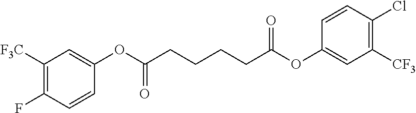 [SMILES: O=C(CCCCC(=O)Oc1ccc(Cl)c(C(F)(F)F)c1)Oc1ccc(F)c(C(F)(F)F)c1] | 1 | 78 | 68 |
| Example 16 | 1.2M LiPF₆ EC/VC/MEC/DEC (29/1/30/40) | 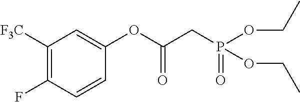 [SMILES: CCOP(=O)(CC(=O)Oc1ccc(F)c(C(F)(F)F)c1)OCC] | 1 | 79 | 61 |
| Example 17 | 1.2M LiPF₆ EC/VC/MEC/DEC (29/1/30/40) | 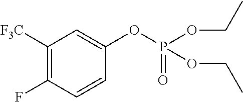 [SMILES: CCOP(=O)(OCC)Oc1ccc(F)c(C(F)(F)F)c1] | 1 | 75 | 62 |
| Comparative Example 1 | 1.2M LiPF₆ EC/VC/MEC/DEC (29/1/30/40) | — | 1 | 62 | 100 |
| Comparative | 1.2M LiPF₆ | 3,4-Diflurophenyl acetate | 1 | 69 | 92 |
| Example 2 | EC/VC/MEC/DEC | | | | |
| | (29/1/30/40) | | | | |
| Comparative | 1.2M LiPF₆ | 4-(Trifluoromethyl)phenyl | 1 | 66 | 89 |
| Example 3 | EC/VC/MEC/DEC | acetate | | | |
| | (29/1/30/40) | | | | |

TABLE 2

| | | Phenyl ester compound | | | |
|---|---|---|---|---|---|
| | | | Addition | Discharge | |
| | | | amount | capacity | Gas |
| | Composition of electrolyte | | (content in | retention | generation |
| | salt | | nonaqueous | rate after | amount |
| | Composition of nonaqueous | | electrolytic | cycle at | after cycle |
| | electrolytic solution | | solution) | 60° C. | at 60° C. |
| | (volume ratio of solvent) | Kind | (% by mass) | (%) | (%) |
| Example 18 | 1.2M LiPF₆ EC/MEC/DEC (30/30/40) | 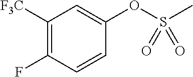 [SMILES: CS(=O)(=O)Oc1ccc(F)c(C(F)(F)F)c1] | 1 | 75 | 73 |
| Example 19 | 1.2M LiPF₆ EC/VC/MEC/DEC (29/1/30/40) | 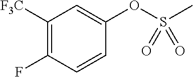 [SMILES: CS(=O)(=O)Oc1ccc(F)c(C(F)(F)F)c1] | 0.05 | 76 | 69 |
| Example 20 | 1.2M LiPF₆ EC/VC/MEC/DEC (29/1/30/40) | 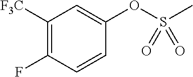 [SMILES: CS(=O)(=O)Oc1ccc(F)c(C(F)(F)F)c1] | 1 | 80 | 64 |
| Example 21 | 1.2M LiPF₆ EC/VC/MEC/DEC (29/1/30/40) | 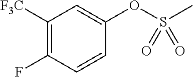 [SMILES: CS(=O)(=O)Oc1ccc(F)c(C(F)(F)F)c1] | 3 | 77 | 60 |
| Example 22 | 1.2M LiPF₆ EC/VC/MEC/DEC (29/1/30/40) | 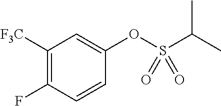 [SMILES: CC(C)S(=O)(=O)Oc1ccc(F)c(C(F)(F)F)c1] | 1 | 78 | 67 |
| Example 23 | 1.2M LiPF₆ EC/VC/MEC/DEC (29/1/30/40) | 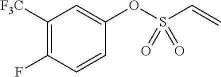 [SMILES: C=CS(=O)(=O)Oc1ccc(F)c(C(F)(F)F)c1] | 1 | 82 | 65 |
| Example 24 | 1.2M LiPF₆ EC/VC/MEC/DEC (29/1/30/40) | 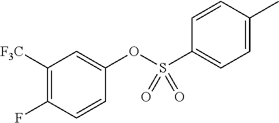 [SMILES: Cc1ccc(S(=O)(=O)Oc2ccc(F)c(C(F)(F)F)c2)cc1] | 1 | 77 | 68 |
| Example 25 | 1.2M LiPF₆ EC/VC/MEC/DEC (29/1/30/40) | 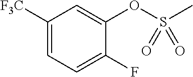 [SMILES: CS(=O)(=O)Oc1cc(C(F)(F)F)ccc1F] | 1 | 79 | 62 |
| Example 26 | 1.2M LiPF₆ EC/VC/MEC/DEC (29/1/30/40) | 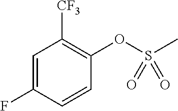 [SMILES: CS(=O)(=O)Oc1ccc(F)cc1C(F)(F)F] | 1 | 79 | 67 |
| Example 27 | 1.2M LiPF₆ EC/VC/MEC/DEC (29/1/30/40) | 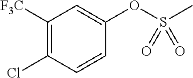 [SMILES: CS(=O)(=O)Oc1ccc(Cl)c(C(F)(F)F)c1] | 1 | 78 | 65 |
| Example 28 | 1.2M LiPF₆ EC/VC/MEC/DEC (29/1/30/40) | 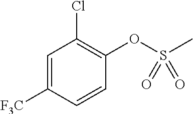 [SMILES: CS(=O)(=O)Oc1ccc(C(F)(F)F)cc1Cl] | 1 | 75 | 69 |
| Example 29 | 1.2M LiPF₆ EC/VC/MEC/PA/DEC (29/1/20/10/40) | 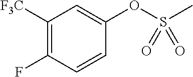 [SMILES: CS(=O)(=O)Oc1ccc(F)c(C(F)(F)F)c1] | 1 | 84 | 66 |
| Example 30 | 1.2M LiPF₆ EC/FEC/MEC/DEC (25/5/50/20) | 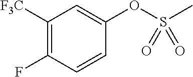 [SMILES: CS(=O)(=O)Oc1ccc(F)c(C(F)(F)F)c1] | 1 | 83 | 75 |
| Example 31 | 1.2M LiPF₆ + 0.05M LiPO₂F₂ EC/VC/MEC/DEC (29/1/30/40) | 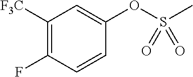 [SMILES: CS(=O)(=O)Oc1ccc(F)c(C(F)(F)F)c1] | 1 | 86 | 65 |
| Example 32 | 1.2M LiPF₆ + 0.05M LiDFOP EC/VC/MEC/DEC (29/1/30/40) | 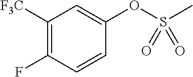 [SMILES: CS(=O)(=O)Oc1ccc(F)c(C(F)(F)F)c1] | 1 | 89 | 63 |
| Example 33 | 1.2M LiPF₆ + 0.05M LiDBOB EC/VC/MEC/DEC (29/1/30/40) | 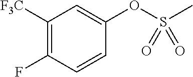 [SMILES: CS(=O)(=O)Oc1ccc(F)c(C(F)(F)F)c1] | 1 | 87 | 67 |

TABLE 3

| | | Phenyl ester compound | | Other compound | | | |
|---|---|---|---|---|---|---|---|
| | Composition of | | Addition | | Addition | Discharge | Gas |
| | electrolyte salt | | amount | | amount | capacity | generation |
| | Composition | | (content in | | (content in | retention | amount |
| | of nonaqueous | | nonaqueous | | nonaqueous | rate after | after |
| | electrolytic solution | | electrolytic | | electrolytic | cycle at | cycle at |
| | (volume ratio | | solution) | | solution) | 60° C. | 60° C. |
| | of solvent) | Kind | (% by mass) | Kind | (% by mass) | (%) | (%) |
| Example 34 Example 35 | 1.2M LiPF₆ EC/VC/MEC/DEC (29/1/30/40) 1.2M LiPF₆ EC/VC/MEC/DEC | 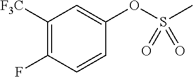 [SMILES: CS(=O)(=O)Oc1ccc(F)c(C(F)(F)F)c1] | 1 1 | 1,6-Diisocyanatohexane Adiponitrile + | 1 0.5 + 0.5 | 83 85 | 60 56 |
| | (29/1/30/40) | | | 2-Methylglutaronitrile | | | |
| Example 36 | 1.2M LiPF₆ | | 1 | 1,3-Dioxane | 0.5 | 83 | 51 |
| | EC/VC/MEC/DEC | | | | | | |
| | (29/1/30/40) | | | | | | |
| Example 37 | 1.2M LiPF₆ | | 1 | 2-Butyne-1,4-diyl | 0.5 | 88 | 63 |
| | EC/VC/MEC/DEC | | | dimethanesulfonate | | | |
| | (29/1/30/40) | | | | | | |
| Example 38 | 1.2M LiPF₆ | | 1 | Vinylsulfonyl fluoride | 0.5 | 90 | 59 |
| | EC/VC/MEC/DEC | | | | | | |
| | (29/1/30/40) | | | | | | |
| Example 39 | 1.2M LiPF₆ | | 1 | 2,4-Butanesultone | 1 | 82 | 58 |
| | EC/VC/MEC/DEC | | | | | | |
| | (29/1/30/40) | | | | | | |
| Example 40 | 1.2M LiPF₆ | | 1 | t-Butylbenzene | 1 | 82 | 62 |
| | EC/VC/MEC/DEC | | | | | | |
| | (29/1/30/40) | | | | | | |

### Example 41 and Comparative Example 4

A positive electrode sheet was produced by using LiNi_{1/2}Mn_{3/2}O₄ (positive electrode active material) in place of the positive electrode active material used in Example 1 and Comparative Example 1. 94% by mass of LiNi_{1/2}Mn_{3/2}O₄ coated with amorphous carbon and 3% by mass of acetylene black (electroconductive agent) were mixed and then added to and mixed with a solution which had been prepared by dissolving 3% by mass of polyvinylidene fluoride (binder) in 1-methyl-2-pyrrolidone in advance, thereby preparing a positive electrode mixture paste. A laminate-type battery was produced and subjected to battery evaluation in the same manners as in Example 1 and Comparative Example 1, except that this positive electrode mixture paste was applied onto one surface of an aluminum foil (collector), dried, and treated under pressure, followed by cutting into a predetermined size, thereby producing a positive electrode sheet; and that in evaluating the battery, the final charging voltage and the final discharging voltage were set to 4.8 V and 2.7 V, respectively. The results are shown in Table 4.

TABLE 4

| | | Phenyl ester compound | | | |
|---|---|---|---|---|---|
| | | | Addition | Discharge | |
| | | | amount | capacity | Gas |
| | Composition of electrolyte | | (content in | retention | generation |
| | salt | | nonaqueous | rate after | amount |
| | Composition of nonaqueous | | electrolytic | cycle at | after cycle |
| | electrolytic solution | | solution) | 60° C. | at 60° C. |
| | (volume ratio of solvent) | Kind | (% by mass) | (%) | (%) |
| Example 41 | 1.2M LiPF₆ EC/FEC/MEC/DEC (25/5/50/20) | 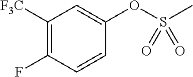 [SMILES: CS(=O)(=O)Oc1ccc(F)c(C(F)(F)F)c1] | 1 | 79 | 78 |
| Comparative | 1.2M LiPF₆ | — | — | 62 | 100 |
| Example 4 | EC/FEC/MEC/DEC | | | | |
| | (25/5/50/20) | | | | |

### Examples 42 and 43 and Comparative Example 5

A negative electrode sheet was produced by using lithium titanate Li₄Ti₅O₁₂ (negative electrode active material) in place of the negative electrode active material used in Example 1 and Comparative Example 1. 80% by mass of lithium titanate Li₄Ti₅O₁₂ and 15% by mass of acetylene black (electroconductive agent) were mixed and then added to and mixed with a solution which had been prepared by dissolving 5% by mass of polyvinylidene fluoride (binder) in 1-methyl-2-pyrrolidone in advance, thereby preparing a negative electrode mixture paste. A laminate-type battery was produced and subjected to battery evaluation in the same manners as in Example 1 and Comparative Example 1, except that this negative electrode mixture paste was applied onto one surface of a copper foil (collector), dried, and treated under pressure, followed by cutting into a predetermined size, thereby producing a negative electrode sheet; and that in evaluating the battery, the final charging voltage and the final discharging voltage were set to 2.7 V and 1.2 V, respectively; and that the composition of the nonaqueous electrolyte was changed to a predetermined composition. The results are shown in Table 5.

TABLE 5

| | | Phenyl ester compound | | | |
|---|---|---|---|---|---|
| | | | Addition | Discharge | |
| | | | amount | capacity | Gas |
| | Composition of electrolyte | | (content in | retention | generation |
| | salt | | nonaqueous | rate after | amount |
| | Composition of nonaqueous | | electrolytic | cycle at | after cycle |
| | electrolytic solution | | solution) | 60° C. | at 60° C. |
| | (volume ratio of solvent) | Kind | (% by mass) | (%) | (%) |
| Example 42 Example 43 | 1.2M LiPF₆ EC/PC/DEC (20/10/70) 1.2M LiPF₆ + LiTFMSB EC/PC/DEC | 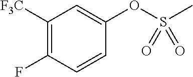 [SMILES: CS(=O)(=O)Oc1ccc(F)c(C(F)(F)F)c1] | 1 1 | 79 83 | 78 73 |
| | (20/10/70) | | | | |
| Comparative | 1.2M LiPF₆ | — | — | 62 | 100 |
| Example 5 | EC/PC/DEC | | | | |
| | (20/10/70) | | | | |

All of the lithium secondary batteries of Examples 1 to 40 as described above are improved in the capacity retention rate after high-temperature cycle and inhibited in the gas generation amount, as compared with Comparative Example 1 which is in the case of not adding the phenyl ester compound and Comparative Examples 2 to 3 which each is in the case of adding other phenyl ester compound than the phenyl ester compound represented by the general formula (I). In the light of the above, it has become clear that the effects brought in the case of using the energy storage device of the invention of the present application over a wide temperature range are peculiar effects brought in the case where the nonaqueous electrolytic solution contains the phenyl ester compound represented by the general formula (I).

In addition, from the comparison of Example 41 with Comparative Example 4 in the case of using lithium nickel manganate (LiNi_{1/2}Mn_{3/2}O₄) for the positive electrode and also from the comparison of Examples 42 and 43 with Comparative Example 5 in the case of using lithium titanate (Li₄Ti₁₅O₁₂) for the negative electrode, the same effects are brought. In consequence, it is evident that the effects of the present invention are not an effect relying upon a specified positive electrode or negative electrode.

Furthermore, the nonaqueous electrolytic solution of the present invention also has effects for improving the discharging properties in the case of using a lithium primary battery at a high temperature and the charging and discharging properties of a lithium ion capacitor.

### INDUSTRIAL APPLICABILITY

The energy storage device using the nonaqueous electrolytic solution of the present invention is useful as an energy storage device, such as a lithium secondary battery, a lithium ion capacitor, etc., each having excellent electrochemical characteristics in the case of using a battery at a high temperature.

1. A nonaqueous electrolytic solution having an electrolyte salt dissolved in a nonaqueous solvent, the nonaqueous electrolytic solution comprising a phenyl ester compound represented by the following general formula (I), in which the benzene ring is substituted with both a halogen atom and a fluoroalkyl group: 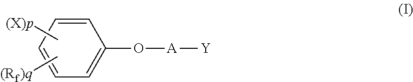
wherein R_{f} represents a fluoroalkyl group having 1 to 6 carbon atoms; X represents a halogen atom; each of p and q is an integer of 1 to 4; (p+q) is 5 or less; A has a structure represented by —S(═O)₂—, —C(═O)—, —C(═O)—O—, —C(═O)-L¹-C(═O)—, —C(═O)-L²-P(═O)(OR)—O—, or —P(═O)(OR)—O—; Y represents a fluorine atom, a hydrogen atom, an alkyl group having 1 to 6 carbon atoms, an alkenyl group having 2 to 6 carbon atoms, an alkynyl group having 3 to 6 carbon atoms, or an aryl group having 6 to 12 carbon atoms; L¹ represents an alkylene group having 1 to 8 carbon atoms, an alkenylene group having 2 to 8 carbon atoms, an alkynylene group having 2 to 8 carbon atoms, or a direct bond; L² represents an alkylene group having 1 to 8 carbon atoms; and R represents an alkyl group having 1 to 6 carbon atoms, provided that only when A is —S(═O)₂—, Y may be a fluorine atom; and only when A is —C(═O)—, Y may be a hydrogen atom, and
wherein at least one hydrogen atom in each group of the alkyl group, the alkenyl group, the alkynyl group, the aryl group, the alkylene group, the alkenylene group, and the alkynylene group may be substituted with a halogen atom.

2. The nonaqueous electrolytic solution according to claim 1, wherein a content of the phenyl ester compound represented by the general formula (I) in the nonaqueous electrolytic solution is 0.01 to 5% by mass in total.

3. The nonaqueous electrolytic solution according to claim 1, wherein the nonaqueous solvent comprises a cyclic carbonate and a linear carbonate, and as the linear carbonate, comprises both a symmetric linear carbonate and an asymmetric linear carbonate.

4. The nonaqueous electrolytic solution according to claim 3, wherein the cyclic carbonate comprises one or more selected from ethylene carbonate, propylene carbonate, 1,2-butylene carbonate, 2,3-butylene carbonate, and a cyclic carbonate having a fluorine atom or an unsaturated bond.

5. The nonaqueous electrolytic solution according to claim 3, wherein the cyclic carbonate having a fluorine atom comprises one or more selected from 4-fluoro-1,3-dioxolan-2-one and trans- or cis-4,5-difluoro-1,3-dioxolan-2-one.

6. The nonaqueous electrolytic solution according to claim 4, wherein the cyclic carbonate having an unsaturated bond comprises one or more selected from vinylene carbonate, vinyl ethylene carbonate, and 4-ethynyl-1,3-dioxolan-2-one.

7. The nonaqueous electrolytic solution according to claim 3, wherein the cyclic carbonate comprises ethylene carbonate or propylene carbonate, and vinylene carbonate or a cyclic carbonate having a fluorine atom.

8. The nonaqueous electrolytic solution according to claim 3, wherein the asymmetric linear carbonate is one or more selected from methyl ethyl carbonate, methyl propyl carbonate, methyl isopropyl carbonate, methyl butyl carbonate, and ethyl propyl carbonate.

9. The nonaqueous electrolytic solution according to claim 3, wherein the symmetric linear carbonate is one or more selected from dimethyl carbonate, diethyl carbonate, dipropyl carbonate, and dibutyl carbonate.

10. The nonaqueous electrolytic solution according to claim 1, wherein the electrolyte salt comprises one or more lithium salts selected from LiPF₆, LiBF₄, LiN(SO₂CF₃)₂, and LiN(SO₂F)₂.

11. An energy storage device comprising a positive electrode, a negative electrode, and a nonaqueous electrolytic solution having an electrolyte salt dissolved in a nonaqueous solvent, the nonaqueous electrolytic solution comprising the phenyl ester compound represented by the general formula (I), in which the benzene ring is substituted with both a halogen atom and a fluoroalkyl group.

12. The energy storage device according to claim 11, wherein an active material of the positive electrode is a complex metal oxide comprising lithium and one or more selected from cobalt, manganese, and nickel, or a lithium-containing olivine-type phosphate comprising one or more selected from iron, cobalt, nickel, and manganese.

13. The energy storage device according to claim 11, wherein an active material of the negative electrode comprises one or more selected from a lithium metal, a lithium alloy, a carbon material capable of absorbing and releasing lithium, tin, a tin compound, silicon, a silicon compound, and a lithium titanate compound.

14. A phenyl ester compound represented by the following general formula (II), in which the benzene ring is substituted with both a halogen atom and a fluoroalkyl group: 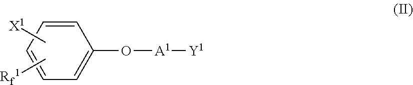
wherein R_{f}¹ represents a fluoroalkyl group having 1 to 6 carbon atoms; X¹ represents a halogen atom; A¹ has a structure represented by —S(═O)₂—, —C(═O)—, —C(═O)—O—, —C(═O)-L³-C(═O)—, —C(═O)-L⁴-P(═O)(OR¹)—O—, or —P(═O)(OR¹)—O—; Y¹ represents a fluorine atom, a hydrogen atom, an alkyl group having 1 to 6 carbon atoms, an alkenyl group having 2 to 6 carbon atoms, an alkynyl group having 3 to 6 carbon atoms, or an aryl group having 6 to 12 carbon atoms; L³ represents an alkylene group having 1 to 8 carbon atoms, an alkenylene group having 2 to 8 carbon atoms, an alkynylene group having 2 to 8 carbon atoms, or a direct bond; L⁴ represents an alkylene group having 1 to 8 carbon atoms; and R¹ represents an alkyl group having 1 to 6 carbon atoms, provided that only when A¹ is —S(═O)₂—, Y¹ may be a fluorine atom; only when A¹ is —C(═O)—, Y¹ may be a hydrogen atom; and the case where A¹ is —S(═O)₂— and Y¹ is a trifluoromethyl group is excluded, and
wherein the alkyl group, the alkenyl group, the alkynyl group, the aryl group, the alkylene group, the alkenylene group, and the alkynylene group, at least one hydrogen atom in each group may be substituted with a halogen atom.

15. The nonaqueous electrolytic solution to claim 1, wherein the phenyl ester compound of formula (I) comprises at least one selected from the group consisting of 4-fluoro-3-(trifluoromethyl)phenyl methanesulfonate, 4-fluoro-3-(trifluoromethyl)phenyl propane-2-sulfonate, 4-fluoro-3-(trifluoromethyl)phenyl vinylsulfonate, 4-fluoro-3-(trifluoromethyl)phenyl 4-methylbenzenesulfonate, 2-fluoro-3-(trifluoromethyl)phenyl methanesulfonate, 4-fluoro-2-(trifluoromethyl)phenyl methanesulfonate, 3-chloro-4-(trifluoromethyl)phenyl methanesulfonate, 4-chloro-3-(trifluoromethyl)phenyl methanesulfonate, 4-fluoro-3-(trifluoromethyl)phenyl acetate, 4-fluoro-3-(trifluoromethyl)phenyl methacrylate, 4-chloro-3-(trifluoromethyl)phenyl acrylate, 4-fluoro-3-(trifluoromethyl)phenyl methyl carbonate, bis(4-fluoro-3-(trifluoromethyl)phenyl) carbonate, 4-chloro-3-(trifluoromethyl)phenyl vinyl carbonate, 4-fluoro-3-(trifluoromethyl)phenyl methyl oxalate, bis(4-fluoro-3-(trifluoromethyl)phenyl) oxalate, bis(4-fluoro-3-(trifluoromethyl)phenyl) succinate, bis(4-fluoro-3-(trifluoromethyl)phenyl) fumarate, bis(4-chloro-3-(trifluoromethyl)phenyl) adipate, 4-fluoro-3-(trifluoromethyl)phenyl 2-(diethoxyphosphoryl)acetate and 4-fluoro-3-(trifluoromethyl)phenyl diethylphosphate.

16. The phenyl ester compound according to claim 14, wherein the phenyl ester compound of formula (II) comprises at least one selected from the group consisting of 4-fluoro-3-(trifluoromethyl)phenyl methanesulfonate, 4-fluoro-3-(trifluoromethyl)phenyl propane-2-sulfonate, 4-fluoro-3-(trifluoromethyl)phenyl vinylsulfonate, 4-fluoro-3-(trifluoromethyl)phenyl 4-methylbenzenesulfonate, 2-fluoro-3-(trifluoromethyl)phenyl methanesulfonate, 4-fluoro-2-(trifluoromethyl)phenyl methanesulfonate, 3-chloro-4-(trifluoromethyl)phenyl methanesulfonate, 4-chloro-3-(trifluoromethyl)phenyl methanesulfonate, 4-fluoro-3-(trifluoromethyl)phenyl acetate, 4-fluoro-3-(trifluoromethyl)phenyl methacrylate, 4-chloro-3-(trifluoromethyl)phenyl acrylate, 4-fluoro-3-(trifluoromethyl)phenyl methyl carbonate, bis(4-fluoro-3-(trifluoromethyl)phenyl)carbonate, 4-chloro-3-(trifluoromethyl)phenyl vinyl carbonate, 4-fluoro-3-(trifluoromethyl)phenyl methyl oxalate, bis(4-fluoro-3-(trifluoromethyl)phenyl) oxalate, bis(4-fluoro-3-(trifluoromethyl)phenyl) succinate, bis(4-fluoro-3-(trifluoromethyl)phenyl)fumarate, bis(4-chloro-3-(trifluoromethyl)phenyl) adipate, 4-fluoro-3-(trifluoromethyl)phenyl 2-(diethoxyphosphoryl)acetate and 4-fluoro-3-(trifluoromethyl)phenyl diethylphosphate.
